(19) United States
(12) Patent Application Publication
(10) Pub. No.: US 2008/0097250 A1
(43) Pub. Date: Apr. 24, 2008

(54) Biological Information Measuring Panel, and Biological Information Measuring Device

(75) Inventors: Masaharu Tochigi, Tochigi (JP); Kazuo Hirose, Tochigi (JP); Shingo Noguchi, Tochigi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Showa Denko K.K., Minato-ku (JP)

(21) Appl. No.: 11/631,603

(22) PCT Filed: Jul. 7, 2005
(86) PCT No.: PCT/JP05/12978

§ 371 Date: Jan. 5, 2007

Related U.S. Application Data
(60) Provisional application No. 60/587,509, filed on Jul. 14, 2004.

(30) Foreign Application Priority Data
Jul. 7, 2004 (JP) 2004-200922

(51) Int. Cl. A61B 5/11; A61G 7/05

(52) U.S. Cl. 600/595

(57) ABSTRACT

A biological information measuring panel 1 has an elastically flexible laying plate portion 2 to be disposed below a subject. A strain detecting sensor D is attached to the laying plate portion 2. Strain changes of the laying plate portion 2 generated in accordance with biological activities of the subject S are detected by the strain detecting sensor D in a state in which the laying plate portion 2 is disposed below the subject S. An output signal from the strain detecting sensor D is used to measure the biological information of the subject S.

### CROSS REFERENCE TO RELATED APPLICATIONS

This application is an application filed under 35 U.S.C. §111(a) claiming the benefit pursuant to 35 U.S.C. §119(e)(1) of the filing date of U.S. Provisional Application No. 60/587,509 filed on Jul. 14, 2004, pursuant to 35 U.S.C. §111(b).

This application claims priority to Japanese Patent Application No. 2004-200922 filed on Jul. 7, 2004 and U.S. Provisional Application No. 60/587,509 filed on Jul. 14, 2004, the entire disclosures of which are incorporated herein by reference in their entireties.

### TECHNICAL FIELD

The present invention relates to a biological information measuring panel used in measuring biological information of a subject, for example, in a lying posture, a biological information measuring mat, a biological information measuring device, and a biological information measuring method.

### BACKGROUND ART

The following description sets forth the inventor's knowledge of related art and problems therein and should not be construed as an admission of knowledge in the prior art.

In medical care facilities or nursing care facilities, for example, for the purpose of monitoring health conditions of subjects such as patients, infants or aged persons, it has been conducted to measure the asleep biological information (e.g., body weight, respiration rate, pulse rate, body movements) of the subject. In recent years, a number of biological information measuring devices for measuring them have been developed (see, JP 2003-552, A; JP 2001-276019, A; JP 2001-46347, A; JP 2000-107154, A; JP H10-216105, A; JP 2003-210434, A, for example). Among these devices, a device having the following structure is known.

In this device, a load cell as a weight sensor is attached to a lower end of each foot of a bed. The entire weight of the bed that a subject lies down is measured by these load cells to thereby measure the subject's biological information based on the measured weight fluctuations (see, JP 2003-552, A).

According to the aforementioned device, since the total load of the subject's weight and the bed's weight is measured by the load cells, by subtracting the bed's weight from the measured total load, the absolute weight of the subject can be measured. However, the above-mentioned device had the following defects.

In the aforementioned device, the biological information of the subject is measured based on the fluctuations of the total load of the subject's weight and the bed's weight as mentioned above. Therefore, as for the respiration rate information and the pulse rate information which are biological information accompanied by slight load changes among various biological activities of the subject, it was difficult to measure them with a high degree of accuracy.

Furthermore, in the aforementioned device, in order to stabilize the measuring accuracy, it is required to increase the rigidity of the entire bed, resulting in an increased weight of the bed and an increased manufacturing cost of the bed.

Furthermore, in the aforementioned device, in order to improve the measuring accuracy, it is necessary to dispose a ball or a roller between the load receiving portion of the load cell and the lower end of the bed foot. This requires precise alignment operation (spin finishing operation) at the time of installing the bed, resulting in a difficult installing operation.

In addition, in the aforementioned device, the type of bed which allows the installation of the load cells is limited. As a result, it is not always possible to install load cells to all of commercially available beds.

The description herein of advantages and disadvantages of various features, embodiments, methods, and apparatus disclosed in other publications is in no way intended to limit the present invention. Indeed, certain features of the invention may be capable of overcoming certain disadvantages, while still retaining some or all of the features, embodiments, methods, and apparatus disclosed therein.

Other objects and advantages of the present invention will be apparent from the following preferred embodiments.

### DISCLOSURE OF INVENTION

The preferred embodiments of the present invention have been developed in view of the above-mentioned and/or other problems in the related art. The preferred embodiments of the present invention can significantly improve upon existing methods and/or apparatuses.

The present invention was made in view of the aforementioned technical background, and aims to provide a biological information measuring panel, a biological information measuring mat, an biological information measuring device, and a biological information measuring method, capable of measuring various biological information of a subject, especially respiration rate information and/or pulse rate information, with a high degree of accuracy and also capable of being applied to almost all of commercially available beds.

The present invention also aims to provide a biological activity monitoring system equipped with the aforementioned panel or mat, capable of assuredly monitoring various biological activities of a subject, especially respiration or pulse status.

To attain the aforementioned objects, the present invention provides the following means.

[1] A biological information measuring panel, comprising:

an elastically flexible laying plate portion to be disposed below a subject; and

a strain detecting sensor attached to the laying plate portion,

wherein strain changes of the laying plate portion generated in accordance with biological activities of the subject are detected by the strain detecting sensor in a state in which the laying plate portion is disposed below the subject, and

wherein an output signal from the strain detecting sensor is used to measure the biological information of the subject.

[2] The biological information measuring panel as recited in the aforementioned Item 1, wherein the laying plate portion is formed into an approximately rectangular belt shape and is to be disposed below the subject so as to extend in a widthwise direction of the subject.

[3] The biological information measuring panel as recited in the aforementioned Item 1 or 2, wherein bending strength of the laying plate portion is set so as to fall within the range of from 7.5×10² to 1.5×10¹² Nmm².

[4] The biological information measuring panel as recited in any one of the aforementioned Items 1 to 3, wherein the laying plate portion is made of a material whose Young's modulus falls within the range of from 3×10⁴ to 30×10⁴ MPa.

[5] The biological information measuring panel as recited in any one of the aforementioned Items 1 to 4, wherein the laying plate portion is set to be 10 to 1,000 mm in length, 300 to 2,000 mm in width, and 0.1 to 30 mm in thickness.

[6] The biological information measuring panel as recited in any one of the aforementioned Items 1 to 5, wherein the laying plate portion is made of aluminum or its alloy.

[7] The biological information measuring panel as recited in any one of the aforementioned Items 1 to 6, further comprising an elastic layer attached to at least one of upper and lower surfaces of the laying plate portion.

[8] The biological information measuring panel as recited in the aforementioned Item 7, wherein the elastic layer is an elastic sheet member, and wherein the elastic sheet member is attached to at least one of upper and lower surfaces of the laying plate portion with a peripheral end portion of the elastic sheet member protruded from a periphery of the laying plate portion.

[9] The biological information measuring panel as recited in any one of the aforementioned Items 1 to 8, wherein the strain detecting sensor is a strain gauge.

[10] The biological information measuring panel as recited in the aforementioned Item 9, wherein at least one strain gauge is attached to at least one of the upper and lower surfaces of the laying plate portion.

[11] The biological information measuring panel as recited in the aforementioned Item 9 or 10,

wherein a pair of strain gauges are attached to facing positions of the upper and lower surfaces of the laying plate portion,

wherein the pair of strain gauges and a pair of dummy resistances are electrically connected each other to form a bridged circuit, and

wherein an output signal from the bridged circuit is used to measure the biological information of the subject.

[12] The biological information measuring panel as recited in the aforementioned Item 9 or 10,

wherein two pairs of strain gauges are attached to facing positions of the upper and lower surfaces of the laying plate portion,

wherein the two pairs of strain gauges are electrically connected each other to form a bridged circuit, and

wherein an output signal from the bridged circuit is used to measure the biological information of the subject.

[13] The biological information measuring panel as recited in any one of the aforementioned Items 9 to 12, wherein the strain gauges are attached to right and left side portions and a central portion of at least one of the upper and lower surfaces of the laying plate portion.

[14] The biological information measuring panel as recited in any one of the aforementioned Items 9 to 13, wherein the laying plate portion is provided with at least one thin thickness portion thinner than the other portion of the laying plate portion and wherein at least one strain gauge is attached to at least one of upper and lower surfaces of the thin thickness portion of the laying plate portion.

[15] The biological information measuring panel as recited in the aforementioned Item 14, wherein the thin thickness portion is formed in the laying plate portion by partially forming a dented portion on at least one of the upper and lower surfaces of the thin thickness portion of the laying plate portion.

[16] The biological information measuring panel as recited in the aforementioned Item 14, wherein the thin thickness portion is formed in the laying plate portion by forming the laying plate portion so that a thickness of the laying plate portion continuously changes in a widthwise direction of the laying plate portion.

[17] The biological information measuring panel as recited in any one of the aforementioned Items 9 to 16, wherein a signal line passing opening for passing input signal lines to the strain gauges and/or output signal lines from the strain gauges is formed near a strain gauge attaching position.

[18] The biological information measuring panel as recited in any one of the aforementioned Items 1 to 8, wherein the strain detecting sensor is a conductive elastomer sensor.

[19] The biological information measuring panel as recited in the aforementioned Item 18, wherein the elastomer sensor is disposed inside the laying plate portion.

[20] The biological information measuring panel as recited in the aforementioned Item 18 or 19, wherein the laying plate portion is divided into an upper plate portion and a lower plate portion, and wherein the upper plate portion and the lower plate portion are secured in a superimposed manner in a state in which the conductive elastomer is sandwiched between the upper plate portion and the lower plate portion.

[21] The biological information measuring panel as recited in any one of the aforementioned Items 1 to 20, further comprising a controlling device for controlling an output signal from the strain detecting sensor is attached to the laying plate portion.

[22] The biological information measuring panel as recited in the aforementioned Item 21, wherein the controlling device is disposed in a dented portion formed in the upper surface or the lower surface of the laying plate portion.

[23] A biological information measuring mat, comprising:

the biological information measuring panel as recited in any one of the aforementioned Items 1 to 22,

wherein the panel is disposed in the mat.

[24] A biological information measuring device, comprising:

the biological information measuring panel as recited in any one of the aforementioned Items 1 to 22 or the biological information measuring mat as recited in the aforementioned Item 23,

wherein biological information of the subject is measured based on an output signal from the strain detecting sensor of the panel or the mat.

[25] The biological information measuring device as recited in the aforementioned Item 24, further comprising:

computing means for computing the biological information of the subject based on the output signal from the strain detecting sensor; and

displaying means for displaying the biological information computed by the computing means.

[26] The biological information measuring device as recited in the aforementioned Item 25, further comprising communicating means configured to transmit the biological information computed by the computing means.

[27] The biological information measuring device as recited in the aforementioned Item 25 or 26, further comprising warning means configured to give a warning based on the biological information computed by the computing means.

[28] A biological information measuring method, comprising the steps of:

preparing the biological information measuring panel as recited in any one of the aforementioned Items 1 to 22 or the biological information measuring mat as recited in the aforementioned Item 23,

detecting strain changes of the laying plate portion of the panel or the mat generated in accordance with biological activities of the subject in a state in which the panel or the mat is disposed below the subject, and

measuring the biological information of the subject based on an output signal from the strain detecting sensor.

[29] The biological information measuring method as recited in the aforementioned Item 28,

wherein the biological information of the subject is computed by computing means based on the output signal from the strain detecting signal, and

wherein the biological information computed by the computing means is displayed by displaying means.

[30] The biological information measuring method as recited in the aforementioned Item 29, wherein the biological information computed by the computing means is transmitted by communicating means.

[31] The biological information measuring method as recited in the aforementioned Item 29 or 30, wherein a warning is given by warning means based on the biological information computed by the computing means.

[32] A biological activities monitoring system, comprising:

the biological information measuring panel as recited in any one of the aforementioned Items 1 to 22, or the biological information measuring mat as recited in the aforementioned Item 23;

computing means configured to compute the biological information of the subject based on an output signal from the strain detecting sensor of the panel or the mat;

displaying means configured to display the biological information computed by the computing means; and

communicating means configured to transmit the biological information computed by the computing means.

Next, the invention as recited in each of the aforementioned Item will be explained together with its function and effects as follows.

In the biological information measuring panel according to the invention as recited in Item [1], the laying plate portion is disposed below the subject. On this laying plate portion, the subject takes a lying posture (e.g., sleeping posture) via a thin upper mat or without such mat. In this state, the strain changes of the laying plate portion generated in accordance with the biological activities of the subject are detected. By this, the detectable limit with respect to the strain changes of the laying plate portion due to the respiration or the pulse among various biological activities of the subject can be improved, and therefore the changes can be detected with a high degree of accuracy. Thus, especially the respiration information and the pulse information among various biological information of the subject can be measured with a high degree of accuracy.

Furthermore, this panel also can detect whether a subject is on the laying portion such as a bed, for example. In other words, this panel can be utilized as an on-bed detecting panel.

Furthermore, in this panel, the panel mounting operation can be completed by simply disposing the laying plate portion below the subject. Therefore, the panel mounting operation can be performed easily, and the panel can be applied to almost all of commercially available beds. Thus, it is excellent in versatility.

In cases where the biological information of a subject lying on a bed when sleeping on the bed is measured, there are the following advantages.

Since the laying plate portion is usually disposed on a mat of a bed at the time of measuring the biological information, even in cases where a person walks around the bed, it is possible to prevent deterioration in detecting accuracy due to the vibrations of the floor on which the bed is mounted. Thus, since it is not necessary to increase the rigidity of the bad, the weight of the bed can be decreased.

In this panel, the laying plate portion is elastically flexible. The material of this laying plate portion is selected in consideration of the bending strength to attain the purpose of this invention. The bending strength is determined by the Young's modulus or the second moment of area. For example, this laying plate portion can be made of metal such as iron, steel, stainless steel, magnesium alloy, aluminum or its alloy, or plastic such as fiber-reinforced plastic.

In this invention, in cases where the biological information of a subject lying on a bed is measured, as mentioned above, the laying plate portion is usually disposed on a mat of a bed. On the other hand, in cases where the biological information of a subject lying on a futon (Japanese style mattress) directly disposed on a tatami or a floor for sleeping is measured, the laying plate portion is usually disposed on the tatami surface or the floor surface, or on the bottom mattress. Accordingly, as the lying surface on which the laying plate portion is disposed, a mat upper surface of a bed, a tatami surface, a floor surface, and a bottom mattress surface can be exemplified. Furthermore, as the lying surface on which the laying plate portion is disposed, a seating surface of a chair or a vehicle seat, and a backrest can be exemplified. Furthermore, in this invention, the laying plate portion can be disposed on a surface, e.g., a toilet seat or bath room floor, on which a subject is sit or comes into contact.

In this invention, as the biological activities of a subject, respiration, a pulse, and body movements (rolling over on bed) can be exemplified.

As for a bed, a bed used by a subject for sleeping (i.e., a bed for sleeping), an examination table, an inspection table, and a stretcher can be exemplified.

In this invention, as the lying state of the subject, for example, a state in which a body of a subject is lying for sleep, break, or health examination can be exemplified.

As the strain changes of the laying plate portion to be detected by the strain detecting sensor, bending strain changes of the laying plate portion, compression strain changes of the laying plate portion and tensile strain changes of the laying plate portion can be exemplified.

Furthermore, in this invention, the strain detecting sensor detects the strain changes of the laying plate portion, such as the bending strain changes of the laying plate portion, the compression strain changes of the laying plate portion, and the tensile strain changes of the laying plate portion. As such a strain detecting sensor, a strain gauge, a conductive elastomer sensor, an optical strain detecting sensor, an electrostriction device sensor, a piezoelectric device sensor and a magnetostrictive device sensor can be exemplified. The strain detecting sensor can be one or two or more sensors selected from the group consisting of the aforementioned sensors.

In the invention as recited in the aforementioned Item [2], the laying plate portion is formed into an approximately rectangular belt shape and is to be disposed below the subject so as to extend in a widthwise direction of the subject. Therefore, the strain of the laying plate portion can be changed assuredly in accordance with the biological activities of the subject. Thus, especially the respiration information and the pulse information among various biological information of the subject can be measured with a higher degree of accuracy.

In the invention as recited in the aforementioned Item [3], the bending strength (i.e., flexural rigidity) of the laying plate portion is set so as to fall within the predetermined range. Therefore, the strain of the laying plate portion changes assuredly in accordance with the biological activities of the subject. Thus, the respiration information and the pulse information of the subject can be measured with a higher degree of accuracy.

In the invention as recited in the aforementioned Item [4], the laying plate portion is made of a material whose Young's modulus falls within the predetermined range. Therefore, the strain of the laying plate portion changes assuredly in accordance with the biological activities of the subject. Thus, the respiration information and the pulse information of the subject can be measured with a higher degree of accuracy.

In the invention as recited in the aforementioned Item [5], the length, the width and the thickness of the laying plate portion are set to fall within the predetermined range respectively. Therefore, the strain of the laying plate portion can be changed assuredly in accordance with the biological activities of the subject. Thus, the respiration information and the pulse information of the subject can be measured with a higher degree of accuracy.

Furthermore, in the invention as recited in the aforementioned Item [5], the following functions and effects can be attained. Since the laying plate portion is set to be 10 to 1,000 mm in length, this laying plate portion can be applied to from a short subject to a tall subject. Since the laying plate portion is set to be 300 to 2,000 mm in width, this laying plate portion can be applied to from an infant to an adult. The reasons that it is preferable to set the thickness of the laying plate portion to 0.1 to 30 mm are as follows. If the thickness of the laying plate portion is 0.1 mm or more, the bending strength required for the laying plate portion can be assuredly set so as to fall within a predetermined range. If the thickness is 30 mm or less, in a state in which the subject takes a lying posture above the laying plate portion, the uncomfortable feeling that a subject may feel by the step due to the thickness of the laying plate portion can be eliminated assuredly.

In the invention as recited in the aforementioned Item [6], since the laying plate portion is made of aluminum or its alloy, the panel can be decreased in weight, enabling an easy installation of the panel.

Furthermore, in this case, the following advantages can also be attained. That is, in general, the Young modulus of aluminum or its alloy is smaller than that of steel such as iron, i.e., the former is about ⅓ of the latter. Accordingly, the strain of the laying plate portion made of aluminum or its alloy can changes very sensitively against the respiration or the pulse of the subject among various biological activities of the subject. Therefore, the respiration information and the pulse information of the subject can be measured with a higher degree of accuracy.

Furthermore, since aluminum or its alloy hardly got rusty, a laying plate portion made of aluminum or its alloy is long in lifetime.

In addition, since aluminum or its alloy is excellent in recyclability, a laying plate portion made of aluminum or its alloy can be recycled at the time of discarding the plate.

In the invention as recited in the aforementioned Item [7], since an elastic layer is attached to at least one of upper and lower surfaces of the laying plate portion, in a state in which the laying plate portion is disposed below the subject, the strain of the laying plate portion can change assuredly. Therefore, the respiration information and the pulse information of the subject can be measured with a high degree of accuracy. Furthermore, this laying plate portion can give a soft feeling to a subject lying on (e.g., sleeping) the laying plate portion. As a result, it is comfortable for the subject.

In the invention as recited in the aforementioned Item [8], since the elastic sheet member (i.e., elastic layer) is attached to at least one of upper and lower surfaces of the laying plate portion with a peripheral end portion of the elastic sheet member protruded from a periphery of the laying plate portion, in a state in which the subject takes a lying posture on the laying plate portion, the uncomfortable feeling that a subject may feel by the peripheral edge can be eliminated assuredly.

In this invention, the elastic sheet member can be attached to the predetermined portion of the laying plate portion using adhesive agent or another mechanical securing means such as caulking, screwing or riveting.

In the invention as recited in the aforementioned Item [9], since the strain detecting sensor is a strain gauge, the strain changes of the laying plate portion can be detected assuredly.

In the invention as recited in the aforementioned Item [10], since at least one strain gauge is attached to at least one of the upper and lower surfaces of the laying plate portion, the strain changes of the laying plate portion can be detected more assuredly.

In this invention, as the attaching means for attaching the strain gauge to the laying plate portion, adhering using adhesive agent and mechanical securing means such as caulking, screwing or riveting can be exemplified.

In the invention as recited in the aforementioned Item [11], the strain changes of the laying plate portion can be detected assuredly.

In this invention, as the dummy resistor, a fixed resistor or a chip resistor, etc can be used.

In the invention as recited in the aforementioned Item [12], the two pairs of strain gauges are electrically connected each other to form a bridged circuit, and an output signal from the bridged circuit is used to measure the biological information of the subject. Therefore, the strain changes of the laying plate portion can be detected assuredly.

In the invention as recited in the aforementioned Item [13], the strain gauges are attached to right and left side portions and a central portion of at least one of the upper and lower surfaces of the laying plate portion. Therefore, the strain of the laying plate portion due to the respiration and pulse of the subject among the biological activities of the subject can be mainly detected by the strain gauge attached to the central portion of the predetermined surface of the laying plate portion. The lying position of the subject on the laying plate portion is detected by the strain gauges attached to the left and right side portions of the predetermined surface of the laying plate portion. Accordingly, the respiration information and the pulse information of the subject can be measured with a high degree of accuracy, and in addition to it, the lying position of the subject can be detected accurately.

In the invention as recited in the aforementioned Item [14], since the laying plate portion is provided with a prescribed thin thickness portion, strain is generated at the thin thickness portion of the laying plate portion in a concentrated manner. By providing at least one strain gauge is attached to at least one surface of the upper and lower surfaces of the thin thickness portion, the strain changes of the laying plate portion can be detected assuredly. Accordingly, the respiration information and the pulse information of the subject can be measured with a high degree of accuracy.

In the invention as recited in the aforementioned Item [15], a predetermined thin thickness portion can be assuredly formed in the laying plate portion.

In the invention as recited in the aforementioned Item [16], a predetermined thin thickness portion can be assuredly formed in the laying plate portion.

In the invention as recited in the aforementioned Item [17], since a prescribed signal line passing opening is formed near a strain gauge attaching position, a desired line layout can be set by passing the signal lines through the opening.

In the invention as recited in the aforementioned Item [18], since the strain detecting sensor is a conductive elastomer sensor, the strain of the laying plate portion can be assuredly detected.

Furthermore, since various conductive elastomers as the conductive elastomer sensor different in shape and size are available, it is possible to mount the sensor over the entire surface of the laying plate portion or at a prescribed limited position of the laying plate portion. That is, using a conductive elastomer sensor as the strain detecting sensor increases the degrees of freedom in selecting the mounting position in the laying plate portion. Moreover, since it is possible to adjust the electric resistance of the conductive elastomer sensor, the strain changes of the laying plate portion can be detected assuredly by setting the electric resistance of the conductive elastomer to a predetermined value.

As the conductive elastomer sensor, a conductive resin sensor and a conductive rubber sensor can be exemplified.

In the invention as recited in the aforementioned Item [19], since the conductive elastomer sensor is disposed inside the laying plate portion, the sensor is protected by the laying plate portion to prevent damages due to outside contacts. As a result, the lifetime of the sensor, i.e., the lifetime of the panel, can be extended.

In the invention as recited in the aforementioned Item [20], the upper plate and the lower plate are secured in a superimposed manner with the conductive elastomer sensor intervened therebetween, and thus the conductive elastomer sensor is disposed inside the laying plate portion. Therefore, the sensor is protected by the laying plate portion, which prevents damages of the sensor due to outside contacts. As a result, the lifetime of the sensor, i.e., the lifetime of the panel, can be extended. Furthermore, the disposing operation (embedding operation) for disposing the conductive elastomer sensor inside the laying plate portion can be performed easily.

In the invention as recited in the aforementioned Item [21], an output signal from the strain detecting sensor can be controlled by the controlling device.

In the invention as recited in the aforementioned Item [22], damages of the controlling device due to the outside contacts can be prevented. As a result, the lifetime of the controlling device, i.e., the lifetime of the panel, can be extended.

In the invention as recited in the aforementioned Item [23], since the biological information measuring mat is provided with the panel according to this invention, the same functions and results as in the panel of this invention can be attained. Furthermore, the panel is disposed inside the mat, and the mat is usually elastic. Accordingly, with this mat, in accordance with biological activities of a subject, the upper surface of the mat vibrates and the vibrations will be transmitted to the laying plate portion, which causes assured strain fluctuations. In other words, the strain fluctuations of the laying plate portion would not be blocked by the mat.

In the invention as recited in the aforementioned Item [24], since the biological information measuring device is provided with the panel or the matt of this invention, the same functions and results as in the panel or mat of this invention can be attained.

In the invention as recited in the aforementioned Item [25], since the biological information measuring device is provided with the prescribed computing means and the prescribed displaying means, the biological information of the subject can be computed assuredly, and the biological information of the subject can be displayed assuredly.

In the invention as recited in the aforementioned Item [26], since the biological information measuring device is provided with communicating means, the biological information of the subject can be measured at a remote place.

In the invention as recited in the aforementioned Item [27], since the biological information measuring device is provided with warning means, if the state of the subject becomes outside an expected circumstance, this can be notified to nurses, care personnel and/or monitoring personnel by an alarm.

In the invention as recited in the aforementioned Item [28], especially the respiration information and the pulse information of the subject among various biological information can be measured with a high degree of accuracy.

In the invention as recited in the aforementioned Item [29], the biological information of the subject can be computed assuredly, and the biological information of the subject can be displayed assuredly.

In the invention as recited in the aforementioned Item [30], the biological information of the subject can be measured at a remote place.

In the invention as recited in the aforementioned Item [31], if the state of the subject becomes outside an expected circumstance, this can be notified to nurses, care personnel and/or monitoring personnel by an alarm.

In the invention as recited in the aforementioned Item [32], especially the state of respiration and pulse of the subject among various biological information can be measured with a high degree of accuracy.

As mentioned above, the effects of the present invention can be summarized as follows.

The biological information measuring panel according to the present invention can detect the strain changes of the laying plate portion due to especially respiration or pulse among various biological activities of the subject with a high degree of accuracy. Accordingly, especially the respiration information and the pulse information among various biological information of the subject can be measured with a high degree of accuracy.

With this panel, the mounting operation can be performed easily, and this panel can be applied to almost all of commercially available bed. It is excellent in versatility. Furthermore, with this panel, in the case of measuring biological information of a subject lying on a bed, there are following advantages.

Furthermore, with is panel, since the laying plate portion is usually disposed on a mat of a bed at the time of measuring the biological information, even in cases where a person walks around the bed, it is possible to prevent deterioration in detecting accuracy due to the vibrations of the floor on which the bed is mounted. Thus, since it is not necessary to increase the rigidity of the bad, the weight of the bed can be decreased.

The biological information measuring mat according to the present invention has the same effects as those of the panel of the present invention.

The biological information measuring device and biological information measuring method according to the present invention can measure especially the respiration information and the pulse information among various biological activities of the subject with a high degree of accuracy.

The biological information measuring system according to the present invention can monitor especially the respiration and the pulse among various biological activities of the subject assuredly.

The above and/or other aspects, features and/or advantages of various embodiments will be further appreciated in view of the following description in conjunction with the accompanying figures. Various embodiments can include and/or exclude different aspects, features and/or advantages where applicable. In addition, various embodiments can combine one or more aspect or feature of other embodiments where applicable. The descriptions of aspects, features and/or advantages of particular embodiments should not be construed as limiting other embodiments or the claims.

### BRIEF DESCRIPTION OF DRAWINGS

The preferred embodiments of the present invention are shown by way of example, and not limitation, in the accompanying figures, in which:

FIG. 1 is a schematic view showing a biological information measuring panel and a biological information measuring device according to a first embodiment of the present invention;

FIG. 2 is a cross-sectional view showing a state in which subject's biological information is being measured using the panel;

FIG. 3A is a partially broken top view of the panel;

FIG. 3B is a partially broken side view of the panel;

FIG. 3C is a bottom view of the panel;

FIG. 4 is an enlarged cross-sectional view taken along the line X-X shown in FIG. 3A;

FIG. 5 is an exploded perspective view showing the panel;

FIG. 6 is a circuit diagram showing a bridged circuit of a strain gage used in the device;

FIG. 7 is a block diagram of the device;

FIG. 8 is a flowchart showing the biographical information measuring processing of the device;

FIG. 9 is a graph showing a measured example of the output signal from the bridged circuit of the device;

FIG. 10A is a perspective view showing a first modified embodiment of a laying plate portion of the panel;

FIG. 10B is a perspective view showing a second modified embodiment of a laying plate portion of the panel;

FIG. 10C is a perspective view showing a third modified embodiment of a laying plate portion of the panel;

FIG. 10D is a perspective view showing a fourth modified embodiment of a laying plate portion of the panel;

FIG. 10E is a perspective view showing a fifth modified embodiment of a laying plate portion of the panel;

FIG. 11A is a partially broken top view showing a biological information measuring panel according to a second embodiment of the present invention;

FIG. 11B is a partially broken side view of the panel;

FIG. 11C is a bottom view of the panel;

FIG. 12 is an exploded perspective view showing the panel;

FIG. 13 is a perspective view showing a biological information measuring mat according to a third embodiment of the present invention;

FIG. 14A is a top view showing a biological information measuring panel according to a fourth embodiment of the present invention;

FIG. 14B is a side view of the panel;

FIG. 14C is a bottom view of the panel; and

FIG. 15 is a modified embodiment of a bridge circuit.

### BEST MODE FOR CARRYING OUT THE INVENTION

In the following paragraphs, some preferred embodiments of the invention will be described by way of example and not limitation. It should be understood based on this disclosure that various other modifications can be made by those in the art based on these illustrated embodiments.

Several preferable embodiments will be explained with reference to drawings.

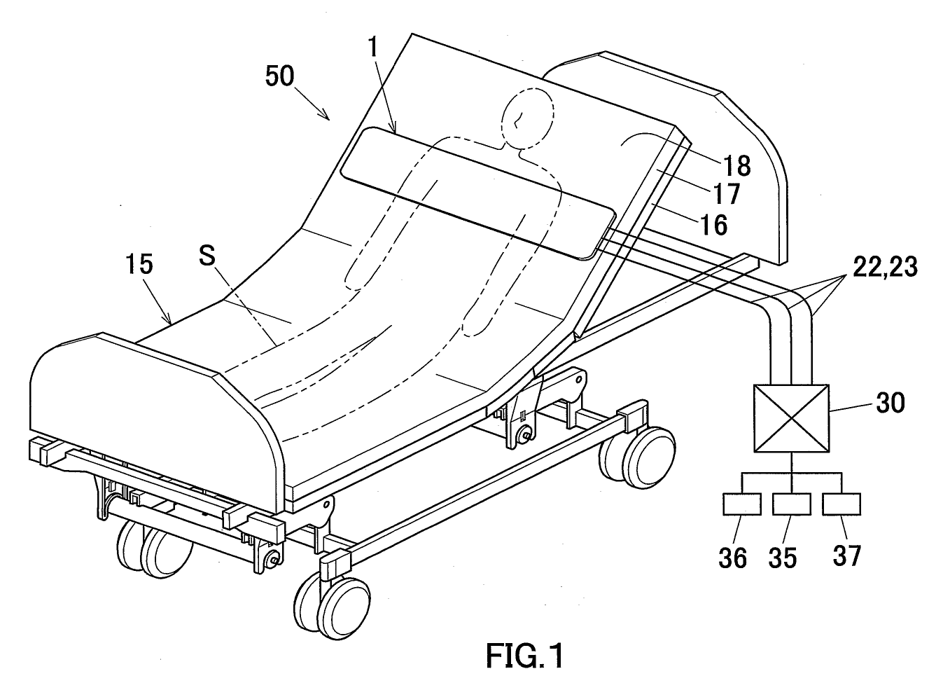

FIGS. 1 to 9 are drawings for explaining a biological information measuring panel 1 and a biological information measuring device 50 according to a first embodiment of the present invention.

The biological information measuring device 50 according to the first embodiment of the present invention is a device to be used in medical care facilities, nursing care facilities or even in general household for monitoring biological information (respiration rate, pulse rate, body movement information) of a subject, especially an asleep subject. As a subject S, a healthy human, a patient, an infant and an aged person can be exemplified.

As shown in FIG. 1, this biological information measuring device 50 is provided with a biological information measuring panel 1, a computing means 30, a displaying means 35, a communicating means 36, and a warning means 37.

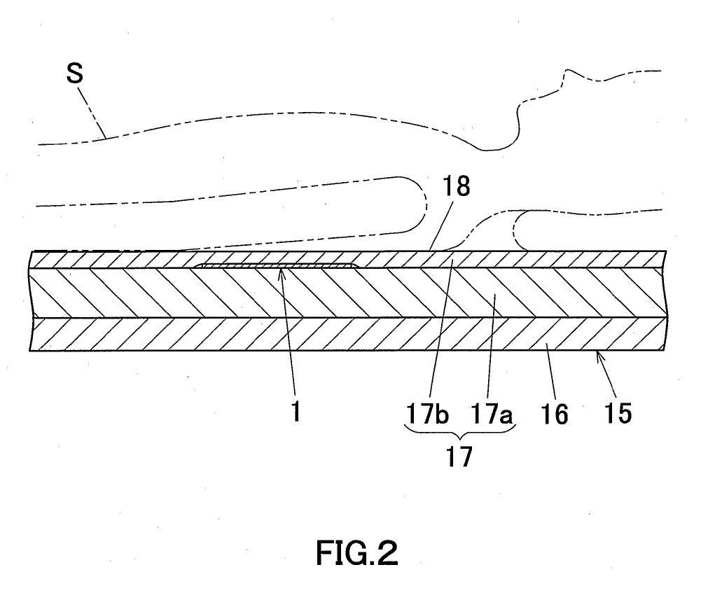

The reference numeral “15” denotes a bed for a subject S. The subject S takes a sleeping posture (i.e., lying posture) on the bed surface 18 of this bed 15. As shown in FIG. 2, the bed surface 18 is an upper surface of an elastic mat (e.g., mattress) 17 disposed on a base plate 16 of the bed 15. The mat 17 is comprised of a thick mat main body 17a and a thin upper mat 17b disposed on the mat main body 17a. In FIG. 1, for the sake of simplicity, the upper mat 17b is not illustrated.

This bed is the so-called “Gatch bed,” and therefore the portion of the base plate 16 which receives the upper body of the subject S can be inclined at a subscribed angle relative to a horizontal plane. At the time of measuring biological information, the entire bed surface 18 is usually disposed horizontally.

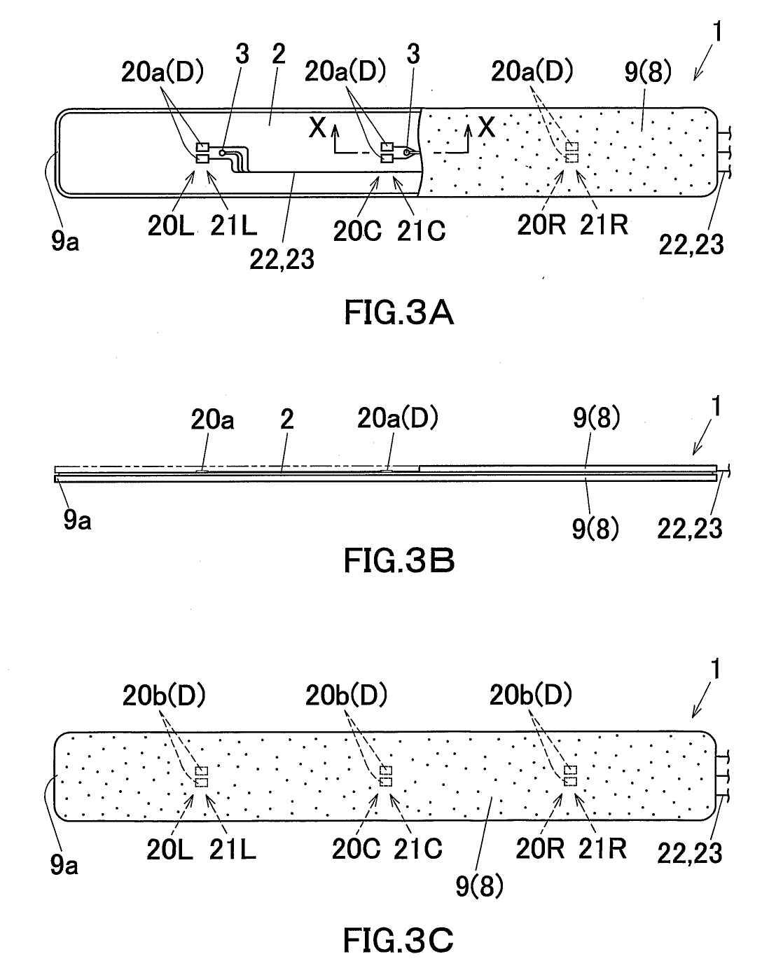

As shown in FIGS. 3 to 5, the panel 1 is provided with a laying plate portion 2, strain sensors D mounted on the laying plate portion 2, and elastic layers 8 attached to the upper and lower surfaces of the laying plate portion 2.

The laying plate portion 2 is an elastic and flexible rectangular band-shaped member. As shown in FIGS. 1 and 2, this laying plate portion 2 is horizontally disposed below the subject S along the widthwise direction of the subject S (i.e., in the widthwise direction of the bed surface 18). In detail, this laying plate portion 2 is disposed horizontally on the upper surface of the mat 17 at a position below the chest region of the lying subject S or therearound so as to extend in the widthwise direction of the subject S.

In this disclosure, for the sake of simplicity, a chest region of a subject S and therearoud will be referred to as a “chest surrounding portion including a chest region of a subject.”

As shown in FIGS. 1 and 2, a subject S is laid down on the central portion of the laying plate portion 2 via the upper mat 17b or without the upper mat 17b. The upper mat 17b plays a role in giving soft feeling to the subject S when the subject S is laid down on the laying plate portion 2. In the present invention, it is not always necessary to use the upper mat 17b.

When a subject S takes a lying posture on the laying plate portion 2, the laying plate portion 2 is elastically slightly bent due to the body weight of the subject S (in detail, the weight of the chest surrounding portion including the chest region of the subject S). This causes flexural strain in the laying plate portion 2. Furthermore, in accordance with various biological activities of the subject S such as the respiration, pulses, body movements (e.g., turning over on bed), the strain of the laying plate portion 2 changes with time. This change is detected by the strain sensor D. The output signal from this strain sensor D will be used for measuring various biological information such as respiration information, pulse information and body movement information (turning over on bed) of the subject S.

In this first embodiment, the laying plate portion 2 is an extruded or rolled member made of aluminum or its alloy.

In the present invention, the laying plate portion 2 can be made of a metallic member made of iron, steel, stainless steel, magnesium alloy, etc. Alternatively, the laying plate portion 2 can be made of a plastic member made of fiber-reinforced plastic.

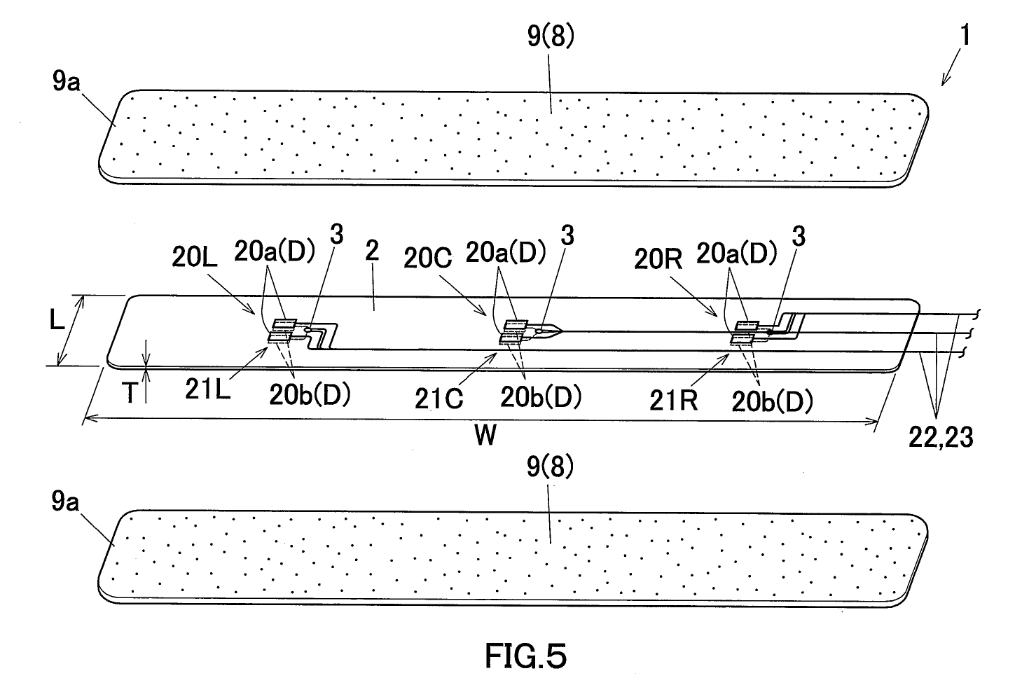

In FIG. 5, “L,” “W,” and “T” denote a length, a width and a thickness of the laying plate portion 2, respectively.

In this disclosure, the length L of the laying plate portion 2 denotes a length of the laying plate portion 2 along the direction of the body height of the subject S, i.e., the length of the laying plate portion 2 along the longitudinal direction of the bed surface 18. The width W of the laying plate portion 2 denotes the length of the laying plate portion 2 along the widthwise direction of the subject S, i.e., the length of the laying plate portion 2 along the widthwise direction of the bed surface 18.

In the panel 1 of this first embodiment, the upper and lower surfaces of the laying plate portion 2 are formed into a flat shape respectively, and the thickness T of the laying plate portion 2 is formed uniformly in the widthwise direction of the laying plate portion 2.

The elastic layer 8 is an elastic sheet member 9 with a predetermined area and thickness. As the elastic sheet member 9, for example, an elastic foamed resin sheet member such as a foamed urethane sheet member or a rubber sheet material can be used. This elastic sheet member 9 is joined to each of upper and lower surfaces of the laying plate portion 2 by adhesive agent with the peripheral edge 9a protruded from the peripheral edge of the laying plate portion 2. Thus, the laying plate portion 2 is provided with elastic layers 8 and 8 on the upper and lower surfaces thereof.

In this first embodiment, as the strain sensor D, a strain gauge 20a, 20a, 20b and 20b is used.

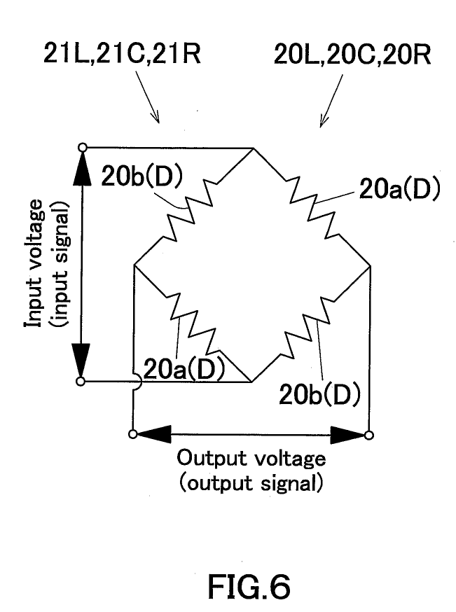

That is, at the facing positions of the upper and lower surfaces of the central portion of the laying plate portion 2, two pairs of strain gauges 20a, 20a, 20b and 20b as strain sensors D are directly attached to the surfaces of the laying plate portion 2. In this disclosure, for the sake of simplicity, the two pairs (i.e., four pieces) of strain gauges 20a, 20a, 20b and 20b will be referred to as “central strain gauge group 20C.” These two pairs of strain gauges 20a, 20a, 20b and 20b are electrically connected with each other as shown in FIG. 6 so as to constitute a bridged circuit 21C (Wheatstone bridge circuit). This bridged circuit 21C will be referred to as a “central bridged circuit 21C,” for the sake of the simplicity.

At the facing positions of the upper and lower surfaces of the left side portion of the laying plate portion 2, two pairs of strain gauges 20a, 20a, 20b and 20b as strain sensors D are directly attached to the surfaces of the laying plate portion 2. For the sake of simplicity, the two pairs (i.e., four pieces) of strain gauges 20a, 20a, 20b and 20b will be referred to as “left side strain gauge group 20L.” These two pairs of strain gauges 20a, 20a, 20b and 20b are electrically connected with each other as shown in FIG. 6 so as to constitute a bridged circuit 21L. This bridged circuit 21L will be referred to as a “left side bridged circuit 21L,” for the sake of the simplicity.

At the facing positions of the upper and lower surfaces of the right side portion of the laying plate portion 2, two pairs of strain gauges 20a, 20a, 20b and 20b as strain sensors D are directly attached to the surfaces of the laying plate portion 2. For the sake of simplicity, the two pairs (i.e., four pieces) of strain gauges 20a, 20a, 20b and 20b will be referred to as “right side strain gauge group 20R.” These two pairs of strain gauges 20a, 20a, 20b and 20b are electrically connected with each other as shown in FIG. 6 so as to constitute a bridged circuit 21R. This bridged circuit 21R will be referred to as a “right side bridged circuit 21R,” for the sake of the simplicity.

Each strain gauge 20a and 20b is attached to a prescribed portion with adhesive agent, for example.

The strain gauges 20a and 20b are used to detect the moment-to-moment strain changes of the laying plate portion 2 which will be generated in accordance with the biological activities of the subject S such as the respiration, pulses, body movements (turning over on bed). On the other hand, the laying plate portion 2 functions as a strain generator.

The output signal from the strain gauges 20a and 20b is used to measure the biological information of the subject S. In this embodiment, since two pairs of strain gauges 20a, 20a and 20b and 20b are electrically connected so as to form a bridged circuit 21L, 21C and 21R, the output voltage as an output signal from each bridged circuit 21L, 21C and 21R is used to measure the biological information of the subject S.

In the present invention, the type of the strain gauge 20a and 20b is not specifically limited. For example, a wire strain gauge, a foil strain gauge or a semiconductor strain gauge can be used as the strain gauge 20a and 20b.

In FIGS. 3 to 5, the reference numeral “22” denotes an input signal line to the strain gauges 20a and 20b, and “23” denotes an output signal line from the strain gauges 20a and 20b. In these figures, the input signal line 22 and the output signal line 23 are shown by a single common line.

In this embodiment, since the two pairs of strain gauges 20a, 20a, 20b and 20b are electrically connected to form a bridged circuit 21L, 21C and 21R, the input signal line 22 to the strain gauges is an input signal line to the bridged circuit 21L, 21C and 21R, and the output signal line 23 from the strain gauges is an output signal line from the bridged circuit 21L, 21C and 21R.

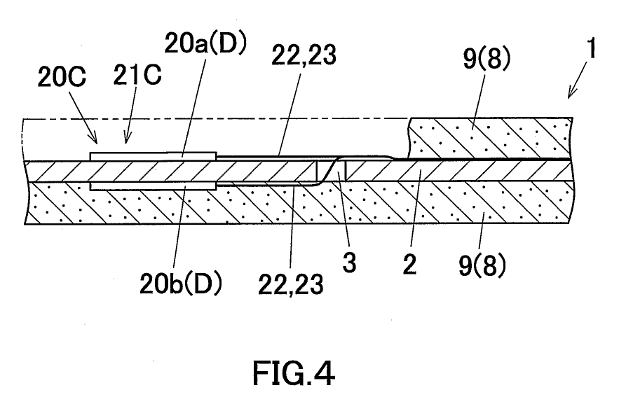

As shown in FIG. 4, at the vicinity of the strain gauge attaching position of each strain gauge group of the laying plate portion 2, a signal line passing hole (opening) 3 is formed. The input signal line 22 to the two strain gauges 20b and 20b attached to the lower surface of the laying plate portion 2 among two pairs (i.e., four pieces) of strain gauges 20a, 20a, 20b and 20b constituting the strain gauge group and the output signal line 23 from the two strain gauges 20b and 20b are introduced to the upper surface side of the laying plate portion 2 via the signal line passing hole 3. Furthermore, these two signal lines 22 and 23, an input signal line 22 to the other two strain gauge 20a and 20a and an output signal line 23 from the other two strain gauge 20a and 20a are extended on the laying plate portion 2 in the right direction of the laying plate portion 2 in a bundled manner and then drawn out of the right edge of the laying plate portion 2.

As explained above, in this panel 1, since the signal line passing hole 3 is formed at the vicinity of the strain gauge attaching position of the laying plate portion 2, any desired wiring layout for the signal lines 22 and 23 can be easily performed by simply passing the signal lines 22 and 23 through the signal line passing hole 3 according to need.

Furthermore, on each the upper and lower surfaces of this laying plate portion 2, the aforementioned elastic sheet member 9 as the elastic layer 8 is attached so as to cover all of the strain gauges 20a and 20b and the signal lines 22 and 23. Thus, the strain gauges 20a and 20b and the signal lines 22 and 23 are protected by the elastic sheet member 9 (i.e., the elastic layer 8), so that damages and/or breaking of the strain gauge 20a and 20b and the signal lines 22 and 23 can be prevented.

Thus, as mentioned above, the laying plate portion 2 is formed into a rectangular band-like shape, and it is to be horizontally disposed below the subject S so as to extend in the widthwise direction of the subject S. By disposing the laying plate portion 2 having the aforementioned configuration as mentioned above, the strain of the laying plate portion 2 changes assuredly in accordance with the biological activities of the subject S. Therefore, among various biological information of the subject S, especially the respiration information and the pulse information can be measured with a high degree of accuracy.

The bending strength (i.e., flexural rigidity) of the laying plate portion 2 is preferably set so as to fall within the range of from 7.5×10² to 1.5×10¹² Nmm². Setting the bending strength within this range causes assured strain changes of the laying plate portion 2 in accordance with the biological activities of the subject S. Therefore, among various biological information of the subject S, especially the respiration information and the pulse information can be measured with a higher degree of accuracy. The more preferable bending strength range is from 7.5×10² to 1.35×10¹² Nmm², still more preferable range is from 1×10⁵ to 7×10⁹ Nmm² (still yet more preferable range is from 7×10⁵ to 2×10⁹ Nmm²).

As to the laying plate portion 2, various types of laying plate portions 2 made of various materials having a fixed length of 100 mm, a width of 300 to 2,000 mm and a thickness of 0.1 to 30 mm were prepared, and the bending strength of each laying plate portion 2 and the S/N ratio of an output signal from each strain gauge 20a and 20b were measured. The results are shown below. The bending strength was calculated by the following formula: [Bending strength]=[“Young's modulus of the material of the laying plate portion 2”×“second moment of area”]

In the case of bending strength: 0.1×10 Nmm²: Average S/N ratio: 20 dB

In the case of bending strength: 7.5×10² Nmm²: Average S/N ratio: 40 dB

In the case of bending strength: 1×10⁵ Nmm²: Average S/N ratio: 40 dB

In the case of bending strength: 1.35×10¹² Nmm²: Average S/N ratio: 40 dB

In the case of bending strength: 1×10¹³ Nmm²: Average S/N ratio: 20 dB

From the results shown above, it could have been confirmed that higher S/N ratio can be attained when the bending strength of the laying plate portion 2 is set so as to fall within the range of from 7.5×10² to 1.5×10¹² (more preferably 1.35×10¹²) Nmm².

The laying plate portion 2 is preferably formed by a material with the Young's modulus falling within the range of 3×10⁴ to 30×10⁴ MPa. In cases where the laying plate portion 2 is formed by the material with the Young modulus falling within this range, the strain of the laying plate portion 2 can change more assuredly in accordance with the biological activities of the subject S. As a result, the respiration information and the pulse information of the subject S can be measured with a higher degree of accuracy. The more preferable range of the Young's modulus is 5×10⁴ to 25×10⁴ MPa.

The laying plate portion 3 is preferably set to 10 to 1,000 mm in length (L), 300 to 2,000 mm in width (W) and 0.1 to 30 mm in thickness (T). In cases where the length L, width W and thickness T are set to fall within the respective range respectively, the strain of the laying plate portion 2 can change more assuredly in accordance with the biological activities of the subject S. As a result, the respiration information and the pulse information of the subject S can be measured with a higher degree of accuracy.

Furthermore, by setting the length L of the laying plate portion 2 so as to fall within the range of 10 to 1,000 mm, this laying plate portion 2 can be applied to from a short subject S to a tall subject S. Thus, the applicable range of this laying plate portion 2 with respect to a subject S can be increased. The more preferable range of the length L is 50 to 500 mm.

By setting the width W of the laying plate portion 2 so as to fall within the range of 300 to 2,000 mm, the laying plate portion 2 can be applied to from infants to adults. Thus, the applicable range of this laying plate portion 2 with respect to a subject S can be increased. The more preferable range of the width W is 300 to 1,500 mm.

The reasons that the thickness T of the laying plate portion 2 is set so as to fall within the range of from 0.1 to 30 mm is as follows. That is, if the thickness T of the laying plate portion 2 is 0.1 mm or more, the bending strength of the laying plate portion 2 can be assuredly set within a prescribed range. If the thickness T of the laying plate portion 2 is 30 mm or less, in a state in which the subject S takes a lying posture on the laying plate portion 2, the uncomfortable feeling that a subject S may feel by the step due to the thickness T of the laying plate portion 2 can be eliminated assuredly. The more preferable range of the thickness T is 0.5 to 10 mm.

The computing means 30 is used to compute the biological information, etc., of the subject S based on the output signal (output voltage) from the strain gauges 20a and 20b. In the first embodiment, since two pairs of strain gauges 20a, 20a, 20b and 20b are electrically connected to form a bridged circuit 21L, 21C and 21R, the computing means 30 computes the biological information of the subject S based on the output signal (output voltage) from the bridged circuit 21L, 21C and 21R.

The displaying means 35 is used to display the biological information of the subject S computed by the computing means 30.

The communication means 36 is used to transmit the biological information, etc., of the subject S computed by the computing means 30 to a remote place or a cellular phone (including PHS).

The warning means 37 is used to issue a warning based on the biological information, etc., of the subject S computed by the computing means 30.

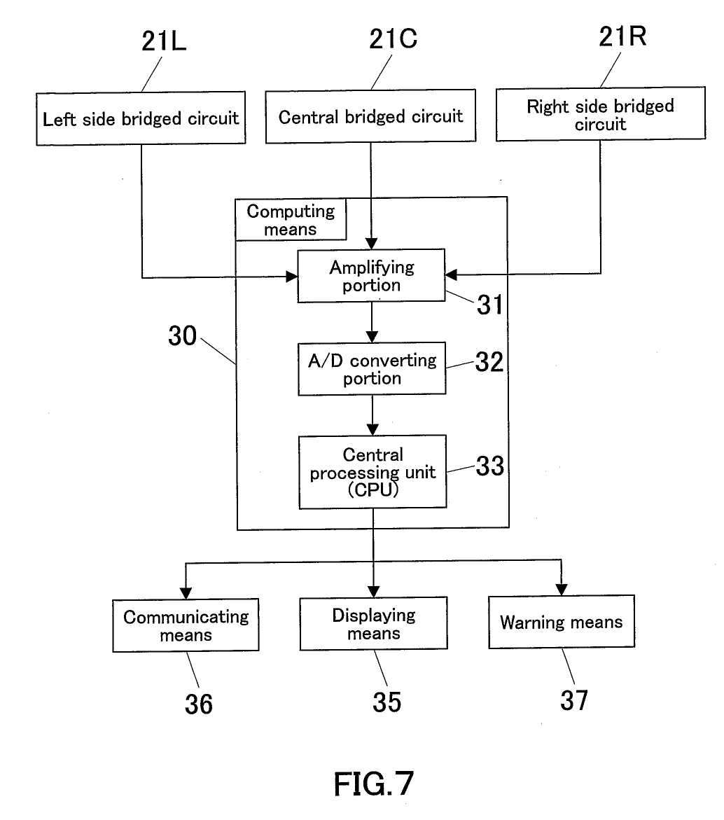

In the computing means 30, as shown in FIG. 7, the output signal (output voltage) from each bridged circuit 21L, 21C and 21R is amplified with the amplifying portion 31, and then converted into a digitalized signal with the A/D converting portion (analog/digital converting portion) 32. Then, this digitalized signal is transmitted to the central processing unit (CPU) 33. In this CPU 33, based on this transmitted digitalized signal, computing on the biological information (respiration information, pulse information, body movement activities, etc.) of the subject S is performed. This computing can be performed by a known method, e.g., a frequency analysis by an FFT method.

Furthermore, in this CPU 33, computing regarding the lying position of the subject S on the laying plate portion 2 is executed. This computing method will be explained briefly as follows. That is, this CPU 33 compares the output signal from the left side bridged circuit 21L with the output signal from the right side bridged circuit 21R using a prescribed calculation formula and computes the lying position of the subject S on the laying plate portion 2 based on the compared result.

Furthermore, in this CPU 33, computing regarding the lying-down-on-bed/leaving-from-bed information of the subject S is also computed.

In the displaying means 35, the biological information (e.g., respiration information, pulse information, body movements) computed by the computing means 30, lying position information, lying-down-on-bed/leaving-from-bed information, etc., of the subject S are displayed on a display such as a monitor television in real time.

The communication means 36 transmits the biological information computed by the computing means 30, lying position information, lying-down-on-bed/leaving-from-bed information, etc., of the subject S to a monitoring room such as a nursing center, a cellular phone, etc., via a certain wired or wireless communication network such as a signal cable, a telephone line, the Internet, a wired LAN, a wireless LAN.

The warning means 37 gives a warning against nurses, the subject, a monitoring person, etc. when the biological information computed by the computing means 30, lying position information, lying-down-on-bed/leaving-from-bed information, etc., of the subject S falls outside a prescribed range.

Next, a biological information measuring method to be performed by using the biological information measuring panel 1 and biological information measuring device 50 according to the first embodiment will be explained as follows.

As shown in FIG. 1, the laying plate portion 2 of the panel 1 is horizontally disposed on a position of the bed surface 18 of the bed 15 corresponding to the chest surrounding portion including a chest region of a subject S in a manner such that the laying plate portion extends in a widthwise direction of the bed surface 18 (i.e., the widthwise direction of the subject S). Furthermore, if necessary, the position of the laying plate portion 2 is fixed so as to be unintentionally shifted with respect to the bed surface 18.

Subsequently, as shown in FIG. 2, if desired, an upper mat 17b is disposed on the laying plate portion 2 so as to cover the entire laying plate portion 2. Thereafter, a subject S lies down on the central portion of the laying plate portion 2. In this state, the chest surrounding portion including the chest portion of the subject S is received by the laying plate portion 2 via the upper mat 17b (or not via the upper mat 17b), so that the laying plate portion 2 is slightly elastically bent by the weight of the chest surrounding portion including the chest portion of the subject S. This causes bending strain at the laying plate portion 2, especially large bending strain at the central portion of the laying plate portion 2.

When the subject S is laid down, the strain of the laying plate portion 2 changes minutely in accordance with the biological activities of the sleeping subject S. This changes are detected by the strain gauges 20a and 20b. The output signal from the strain gauge 20a and 20b, i.e., the output signal from the bridged circuit 21L, 21C and 21R, is transmitted to the computing means 30 via the output signal lines 23. Based on the transmitted output signal, the biological information, lying position information, etc., of the subject S are calculated by the computing means 30.

The biological information, lying position information, etc., of the subject S calculated by the computing means 30 are displayed on the displaying means 35 and transmitted by the communication means 36. Furthermore, if the biological information, lying position information, etc., of the subject S falls outside a predetermined range, a warning is given by the warning means 37.

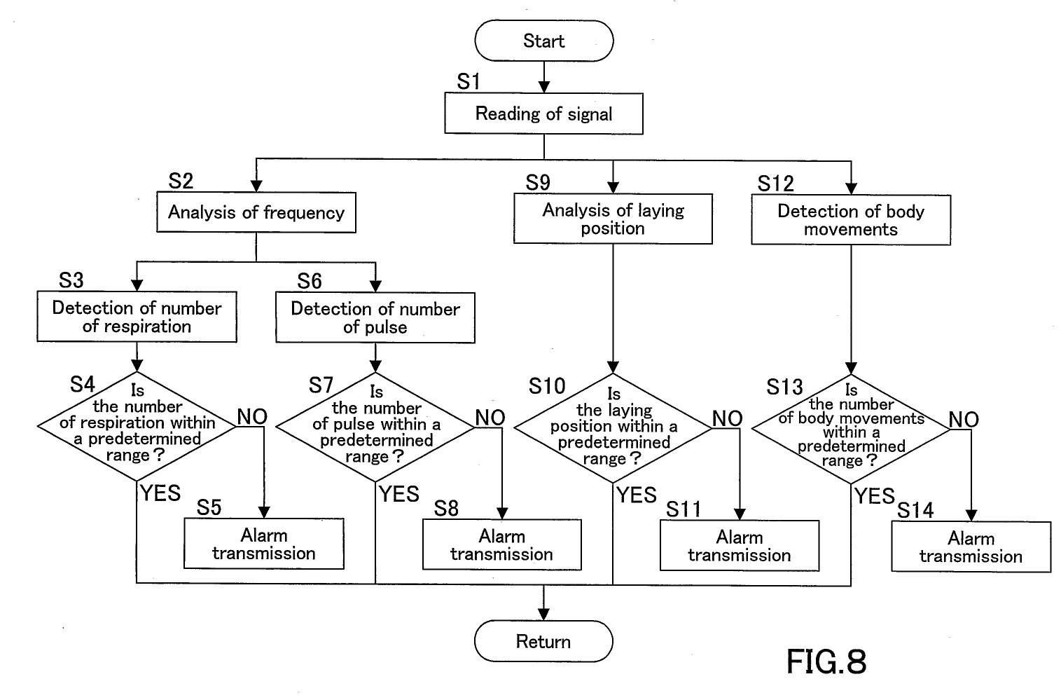

Next, the processing to be executed by the CPU 33 of the computing means 30 will be explained with reference to FIGS. 7 and 8 as follows.

Previously stored in the computing means 30 are a preferable range of each of the respiration rate, the pulse rate and the number of body movements and a preferable lying position of the subject S on the laying plate portion 2.

As shown in FIG. 7, the output signals from three bridged circuits 21L, 21C and 21R are amplified by the amplifying portion 31 respectively as mentioned above, and then converted into digital signal at the A/D converting portion. This digital signal is transmitted to the CPU 32.

As shown in FIG. 8, in the CPU 33, the three signals transmitted are read at Step S1. Then, the routine proceeds to Steps S2, S9 and S12.

At Step 2, based on the output signal from the central bridged circuit 21C among three signals, a frequency analysis is executed by a known method such as an FFT method. Then, the routine proceeds to Steps S3 ad S6.

At Step S3, the respiration rate is detected based on the analyzed frequency. Then, the routine proceeds to Step S4.

At Step S4, it is discriminated whether the respiration rate falls within a predetermined range. If it is discriminate that the respiration rate falls outside the predetermined range (NO at S4), the routine proceeds to Step S5 to give a warning signal to the warning means 37. On the other hand, if it is discriminated that the respiration rate falls within the predetermined range (YES at S4), the routine returns to Step S1. In this Step S4, for example, the respiration information (including snoring information) of the subject S, and information on the life-or-death of the subject, the sleeping state, the apnea syndrome during sleeping, etc., can be obtained.

At Step S6, the pulse rate is detected from the analyzed frequency, and then the routine proceeds to Step S7.

At Step S7, it is discriminated whether the pulse rate falls within a predetermined range. If it is discriminate that the pulse rate falls outside the predetermined range (NO at S7), the routine proceeds to Step S8 to give a warning signal to the warning means 37. On the other hand, if it is discriminated that the pulse rate falls within the predetermined range (YES at S7), the routine returns to Step S1. In this Step S7, for example, information on the life-or-death of the subject, the sleeping state, etc., can be obtained.

At Step S9, the output signal from the left side bridged circuit 21L and the output signal from the right side bridged circuit 21R among three signals are compared using a prescribed computational expression. Based on the compared result, an analysis on the sleeping position of the subject S on the laying plate portion 2 is performed. Then, the routine proceeds to Step S10.

At Step S10, it is discriminated whether the lying position of the subject S is a predetermined position (e.g., the lying position is a central portion of the laying plate portion 2). If it is discriminate that the lying position of the subject S is not a predetermined position (e.g., the lying position is the left end portion or the right end portion of the laying plate portion 2) (NO at S10), the routine proceeds to Step S11 to give a warning signal to the warning means 37. Based on the warning given by the warning means 37, an appropriate measure for preventing the subject S from falling down from the bed surface 18 can be performed. On the other hand, if it is discriminated that the lying position is the predetermined position (YES at S10), the routine returns to Step S1.

At Step S12, detection of the body movements of the subject S such as the rolling over on bed is performed. Then, the routine proceeds to S13.

At Step S13, it is discriminated whether the number of body movements of the subject S such as the number of rolling over falls within a predetermined range. If it is discriminated that the number of body movements falls outside the predetermined range (NO at S14), the routine proceeds to Step S14 to give a warning signal to the warning means 37. Based on the warning given by the warning means 37, an appropriate measure for preventing bedsores from occurring can be performed. On the other hand, if it is discriminated that the number of body movements of the subject S falls within the predetermined range (e.g., the number of body movements are appropriate) (YES at S14), the routine returns to Step S1.

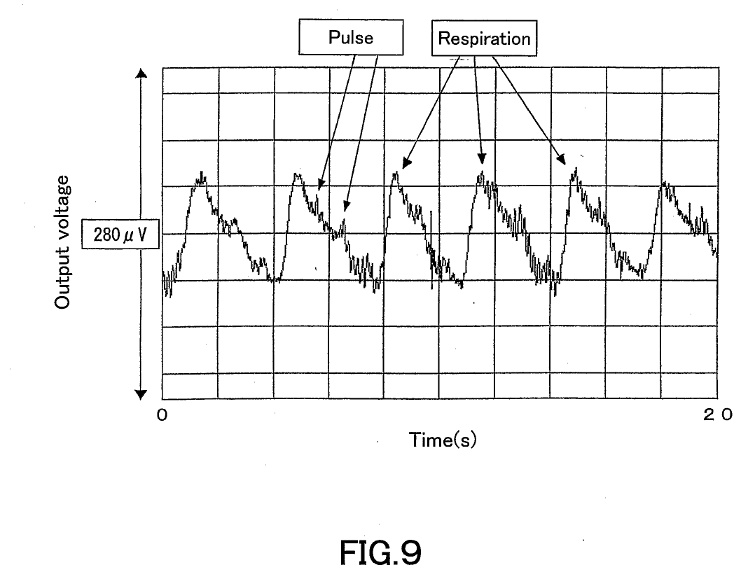

FIG. 9 is a drawing (graph) showing an example of an output signal (output voltage) from the central bridged circuit 21C in the case where biological information of a subject S was actually measured using the biological information measuring panel 1 and the biological information measuring device 50 according to the first embodiment.

In the biological information measuring panel 1 used in this measuring, the material of the laying plate portion 2 was JIS (Japanese Industrial Standard) A6061-T6, the Young's modulus was 7×10⁴ MPa, the length L was 100 mm, the width W was 800 mm, the thickness T was 2 mm, the second moment of area was 533 mm⁴, and the bending strength was 3.7×10⁷ Nmm².

In this figure, the output signal from the bridged circuit 21C is shown as a waveform. In this waveform, the respiration wave components due to the respiration of the subject S and the pulse wave components due to the pulse of the subject S are overlapped. In this output waveform, the continuous wavy waveform with a long wavelength denotes the respiration wave component, and the pulse waveform denotes the pulse wave component. In this output waveform, the breathing cycle was about 3 to 4 seconds, and the pulse cycle was about 1 seconds.

As shown in FIG. 9, according to the biological information measuring panel 1 and the biological information measuring device 50 according to the first embodiment, the respiration wave component and the pulse wave component can be detected clearly and assuredly. Accordingly, it is possible to measure, with a high degree of accuracy, the respiration information (the number of respiration) and the pulse information (the number of pulse) which are biological information with only slight load changes and were difficult to measure with a conventional biological information measuring device among various biological information of the subject S.

Furthermore, the biological information measuring panel 1 and the device 50 according to the first embodiment have the following advantages.

According to the biological information measuring panel 1 of the first embodiment, the installation of the panel 1 can be completed by simply disposing the laying plate portion 2 on the bed surface 18. Therefore, the installation of the panel 1 can be performed easily, and the panel 1 can be applied to almost all of the commercially available beds 15.

Furthermore, since the laying plate portion 2 is disposed on the bed surface 18 at the time of measuring the biological measuring, it is possible to prevent deterioration in detection accuracy due to possible vibrations of the floor on which the bed 15 is installed even if a person walks around the bed 15. Furthermore, since it is not necessary to enhance the rigidity of the bed 15, the bed 15 can be, decreased in weight.

The laying plate portion 2 is formed into a rectangular band shape and is to be horizontally disposed below the subject S along the widthwise direction of the subject S, the changes of strained the laying plate portion 2 due to the respiration or pulse of the subject Scan be assuredly detected. Accordingly, the respiration information or pulse information of the subject S can be measured more accurately.

Furthermore, since the laying plate portion 2 is made of aluminum or its alloy, the following advantages can be expected.

That is, the laying plate portion 2 can be decreased in weight, enabling an easy installation of the panel 1.

In general, the Young modulus of aluminum or its alloy is smaller than that of steel such as iron, i.e., the former is about ⅓ of the latter. Accordingly, the strain of the laying plate portion 2 made of aluminum or its alloy can be changed very sensitively against the respiration or the pulse of the subject S among various biological activities of the subjects. Therefore, the respiration information and the pulse information of the subject S can be measured with a higher degree of accuracy.

Furthermore, since aluminum or its alloy hardly got rusty, a laying plate portion 2 made of aluminum or its alloy is long in lifetime.

In addition, since aluminum or its alloy is excellent in recyclability, a laying plate portion 2 made of aluminum or its alloy can be recycled at the time of discarding the laying plate portion 2.

In this panel 1, two pairs of strain gauges 20a, 20a, 20b and 20b are electrically connected to form a bridged circuit 21L, 21C and 21R, and the output signal from this bridged circuit 21L, 21C and 21R is used to measure the biological information of the subject S. Therefore, the slight changes in strain of the laying plate portion 2 can be detected assuredly. Accordingly, the respiration information and the pulse information of the subject S can be measured with a higher degree of accuracy.

Furthermore, the strain gauges 20a and 20b are attached to the upper and lower surfaces of the right and left side portions and the central portion of the laying plate portion 2. Therefore, the vibrations due to the respiration and pulse of the subject S among the biological activities of the subject S can be detected by the central bridged circuit 21C. The lying position of the subject S on the laying plate portion 2 is detected by the left side bridged circuit 21L and the right side bridge circuit 21R. Accordingly, the respiration information and the pulse information of the subject S can be measured with a high degree of accuracy, and in addition to it, the lying position of the subject S can be detected accurately.

Furthermore, this panel 1 can detect whether a subject S is on the bed surface 18 by comparing the output signal from the strain gauges 20a and 20b with the on-bed judgment standard set value previously stored in the storing portion (not shown) of the computing means 30. In other words, this panel 1 can also be used as an on-bed detecting panel.

The biological information measuring device 50 according to the first embodiment is provided with the computing means 30 and the displaying means 35, and therefore the biological information of the subject S can be computed assuredly and that the biological information of the subject S can be displayed assuredly.

Moreover, since the biological information measuring device 50 is provided with the communicating means 36, the biological information of the subject S can be measured at a remote place.

Furthermore, since the biological information measuring device 50 is provided with the warning means 37, if the state of the subject S becomes outside an expected circumstance, this can be notified to nurses, care personnel and/or monitoring personnel by an alarm.

Furthermore, the biological activities monitoring system of the first embodiment is provided with the biological information measuring panel P1, the computing means 30, the displaying means 35, the communicating means 36, and the warning means 37. Accordingly, with this monitoring system, the status of the respiration and pulse among various biological activities of the subject S can be monitored more assuredly.

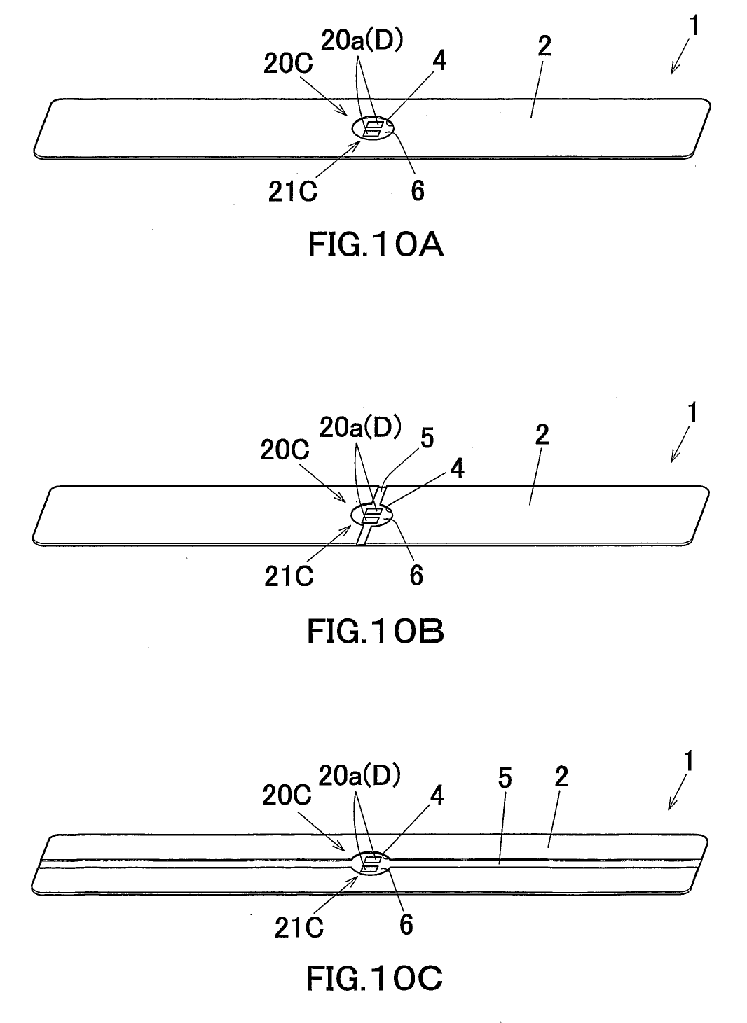

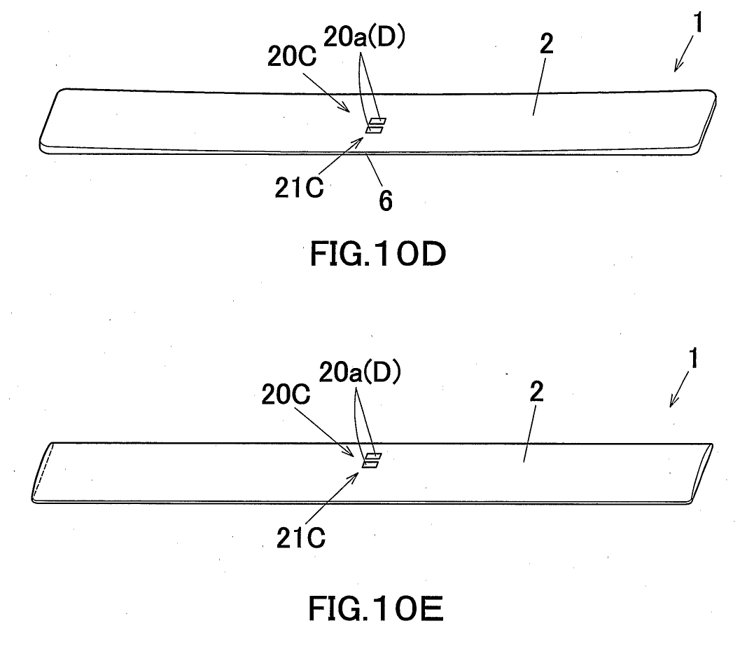

FIGS. 10A to 10E show the first to fifth modified embodiments of the laying plate portion 2 of the panel 1. In these figures, an elastic layer 8 (i.e., elastic sheet member 9) is not illustrated.

In the first embodiment shown in FIG. 10A, at the central portion on the upper and lower surfaces of the laying plate portion 2, a dented portion 4 of a round shape is formed partially. Thus, only at the central portion of this laying plate portion 2, a thin thickness portion 6 thinner in thickness relative to the remaining widthwise portion of this laying plate portion 2 is formed. On the bottom surface of each dented portion 4 formed in the upper and lower surfaces of the thin thickness portion 6, strain gauges 20a and 20b constituting a central strain gauge group 20C are attached.

In the second embodiment shown in FIG. 10B, at the central portion on the upper and lower surfaces of the laying plate portion 2, a dented portion 4 of a round shape and a groove 5 crossing the dented portion 4 and extending in the longitudinal direction of the laying plate portion 2 are formed partially. Thus, only at the central portion of this laying plate portion 2, a thin thickness portion 6 thinner in thickness relative to the remaining widthwise portion of this laying plate portion 2 is formed. On the bottom surface of each dented portion 4 formed in the upper and lower surfaces of the thin thickness portion 6, strain gauges 20a and 20b constituting a central strain gauge group 20C are attached.

In the third embodiment shown in FIG. 10C at the central portion on the upper and lower surfaces of the laying plate portion 2, a dented portion 4 of a round shape and a groove 5 crossing the dented portion 4 and extending in the widthwise direction of the laying plate portion 2 are formed partially. Thus, only at the central portion of this laying plate portion 2, a thinner thickness portion 6 thinner in thickness relative to the remaining widthwise portion of this laying plate portion 2 is formed. On the bottom surface of each dented portion 4 formed in the upper and lower surfaces of the thinner thickness portion 6, strain gauges 20a and 20b constituting a central strain gauge group 20C are attached.

In the fourth embodiment shown in FIG. 10D, the lower surface of the laying plate portion 2 is formed into a flat shape. On the other hand, the upper surface of the laying plate portion 2 is curved into a cylindrical peripheral shape along the entire length such that the central portion in the widthwise direction of the laying plate portion 2 is lowest in height. Thus, the thickness of this laying plate portion 2 continuously changes so that the central portion becomes the thinnest thickness portion 6 in the widthwise direction of the laying plate portion 2. In other words, the thickness of the central portion of this laying plate portion 2 is thinner than the right and left side portions of the laying plate portion 2. Thus, only at the central portion of this laying plate portion 2, a thin thickness portion 6 thinner in thickness than the remaining widthwise portion of the laying plate portion 2 is formed. On each of the upper and lower surfaces of this thin thickness portion 6, strain gauges 20a and 20b constituting a central gauge group 20C is attached.

Thus, in the first to fourth modified embodiments shown in FIGS. 10A to 10 D, the thin thickness portion 6 is formed at the central portion of the laying plate portion 2. Accordingly, in a state in which the laying plate portion 2 is disposed below the subject S, strain will be generated at the thin thickness portion 6 in a concentrated manner. Thus, by attaching strain gauges 20a and 20b on the upper and lower surfaces of such a thin thickness portion 6 of the laying plate portion 2, the changes in strain of the laying plate portion 2 can be detected more assuredly. Therefore, the respiration information or the pulse information of the subject S can be measured assuredly.

In the aforementioned first to fourth modified embodiments, the thin thickness portion 6 is formed only at one portion of the laying plate portion 2, however, the present invention allows to provide two or more thin thickness portions in the laying plate portion 2.

In the fifth embodiment shown in FIG. 10E, the laying plate portion 2 is formed into a D-shape in cross-section with an upwardly expanded surface. In this laying plate portion 2, the thickness is set to be uniform along the widthwise direction of this laying plate portion 2. On the upper and lower surfaces of the central portion of the laying plate portion 2, strain gauges 20a and 20b constituting a central gauge group 20C are attached.

In the present invention, the configuration of the laying plate portion 2 is not limited to the first to fifth embodiments and can be changed arbitrarily.

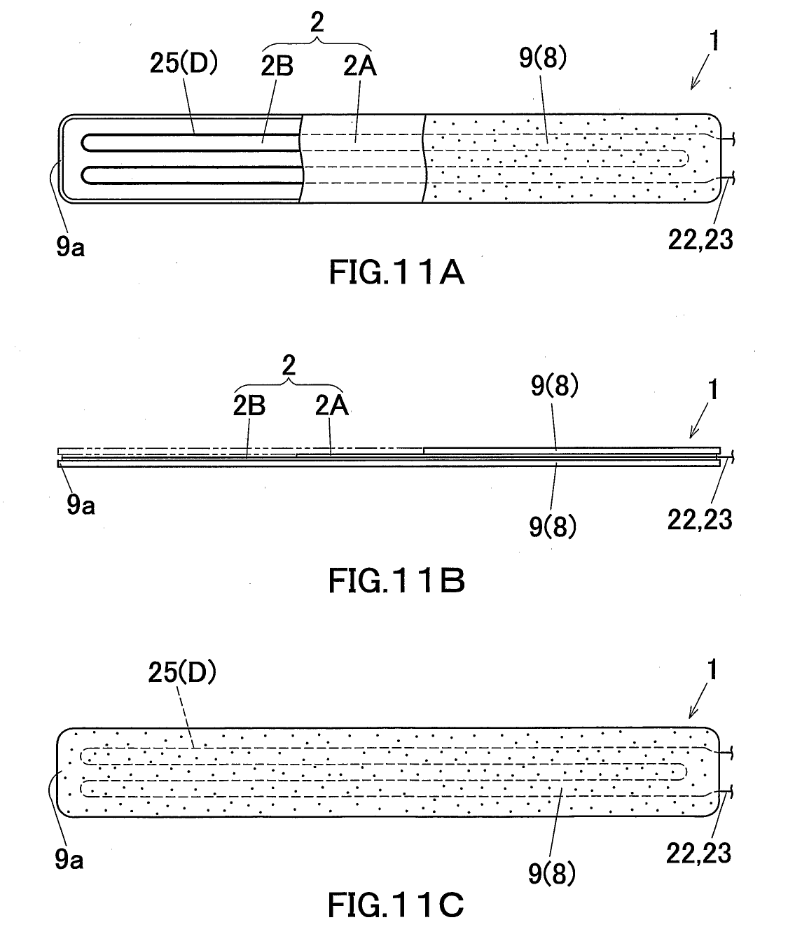

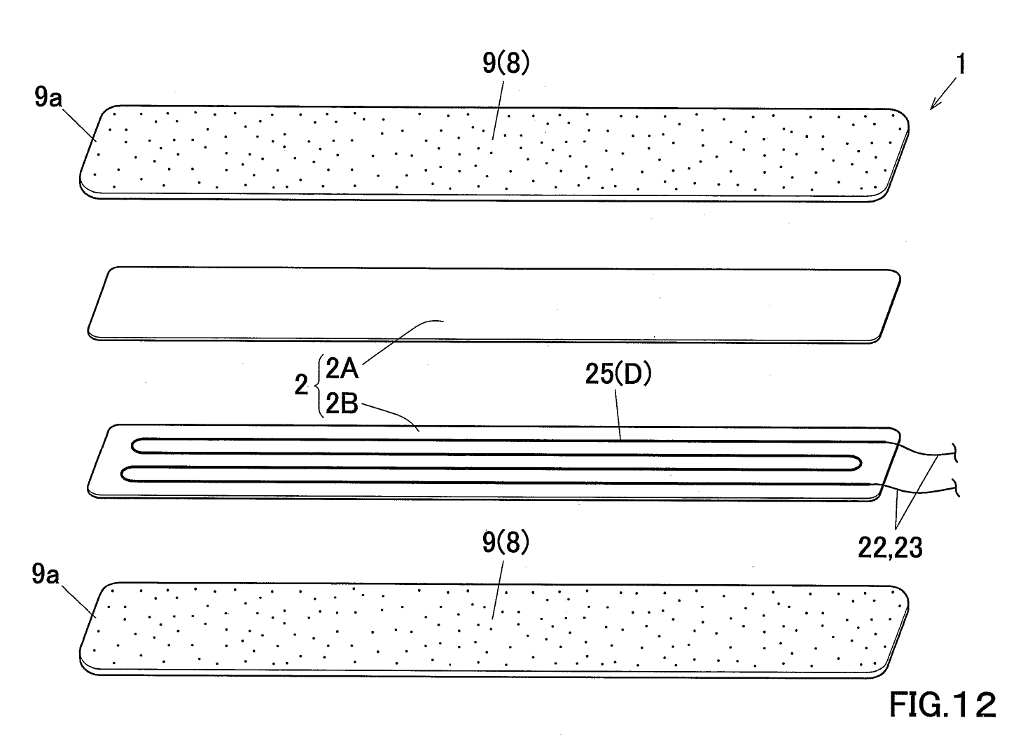

FIGS. 11 and 12 are drawings for explaining a biological information measuring panel 1 according to the second embodiment of this invention.

In this second embodiment, a conductive rubber sensor 25 such as a pressure sensitive conductive rubber sensor is used as the strain detecting sensor D. This conductive rubber sensor 25 is embedded in the laying plate portion 2. The structure of this panel 1 will be explained as follows.

In this panel 1, the laying plate portion 2 is divided into an upper plate 2A and a lower plate 2B. These upper and lower plates 2A and 2B are secured in a superimposed manner with a wire-like-conductive rubber sensor 25 intervened therebetween. This conductive rubber sensor 25 is disposed approximately on the entire area of the superimposing surfaces of the upper and lower plates 2A and 2B in a zigzag manner.

As a means for securing the upper plate 2A and the lower plate 2B, adhesive agent or welding can be exemplified. In place of the above, any mechanical securing means including caulking, clipping and riveting can also be used.

Another structure of the panel 1 and biological information measuring device of this second embodiment and the biological information measuring method are the same as those of the aforementioned first embodiment.

In this panel 1 of the second embodiment, since the strain detecting sensor D is the conductive rubber sensor 25, the strain changes of the laying plate portion 2 can be detected assuredly.

Furthermore, since various conductive rubbers as the conductive rubber sensor 25 different in shape and size are available, it is possible to mount the sensor 25 over the entire surface of the laying plate portion 2 or at a prescribed limited position of the laying plate portion 2. That is, using a conductive rubber sensor 25 as the strain detecting sensor D increases the degrees of freedom in selecting the mounting position in the laying plate portion 2. Moreover, since it is possible to adjust the electric resistance of the conductive rubber sensor 25, the strain changes of the laying plate portion 2 can be detected assuredly by setting the electric resistance of the conductive rubber to a predetermined value.

Furthermore, as mentioned above, the upper plate 2A and the lower plate 2B are secured in a superimposed manner with the conductive rubber sensor 25 intervened therebetween, and thus the conductive rubber sensor 25 is disposed, inside the laying plate portion 2. Therefore, the sensor 25 is protected by the laying plate portion 2, which prevents damages of the sensor due to outside contacts. As a result, the lifetime of the sensor, i.e., the lifetime of the panel 1, can be extended. Furthermore, since the conductive rubber sensor 25 is disposed inside the laying plate portion 2 due to the securing operation of the upper and lower plates 2A and 2B, the disposing operation (embedding operation) for disposing the conductive rubber sensor 25 inside the laying plate portion 2 can be performed easily.

In the second embodiment, although the conductive rubber of the conductive rubber sensor 25 is a wire-shaped member, in this invention, it can be a sheet-shaped member such as a belt-like member, or can be any other shape.

In this invention, in place of the conductive rubber sensor 25, a conductive resin sensor can be used as a conductive elastomer sensor.

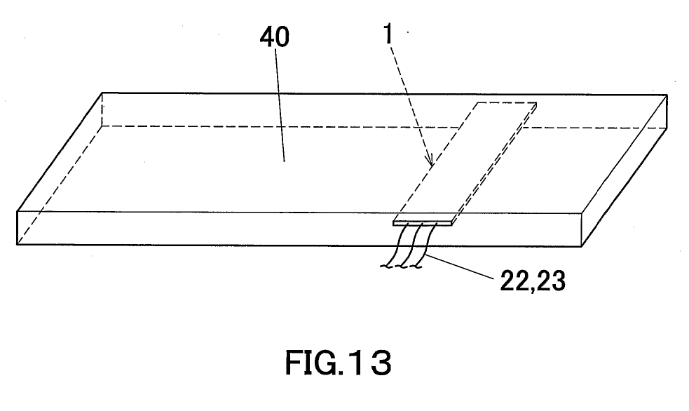

FIG. 13 is an explanatory view of a biological information measuring mat 40 according to the third embodiment of this invention.

This mat 40 according to the third embodiment is provided with the biological information measuring panel 1 of the first embodiment. The structure of this mat 40 will be explained below.

The mat 40 can also be used as a mat 17 (e.g., mattress) to be disposed on a base plate portion 16 of a bed 15 (see, FIGS. 1 and 2), or a mat (e.g., bottom mattress) to be disposed on a tatami or a floor, and thus is formed to have a size capable of accommodating a subject S. In this mat 40, an elastic cushion material is filled in it, and therefore this mat 40 has elasticity.

At the thickness central portion (in detail, mat upper surface vicinity portion of the thickness central portion) of the portion receiving the chest surrounding portion including a chest of a subject S, the panel 1 is disposed horizontally in an embedded state so as to extend in the widthwise direction of the mat 40. The width of the mat 40 is set to be almost the same as the width of the panel 1.

In this third embodiment, since the panel 1 according to the first embodiment is disposed inside the mat 40, when a subject S is laid on the mat 40, the upper surface of the mat 40 vibrates slightly. Since these vibrations will be transmitted to the laying plate portion 2 without being attenuated, the strain of the laying plate portion 2 can change assuredly, enabling an accurate measurement of the respiration information and the pulse information of the subject S.

Another structure of the mat 40 and biological information measuring device of this third embodiment and the biological information measuring method are the same as those of the aforementioned first embodiment.

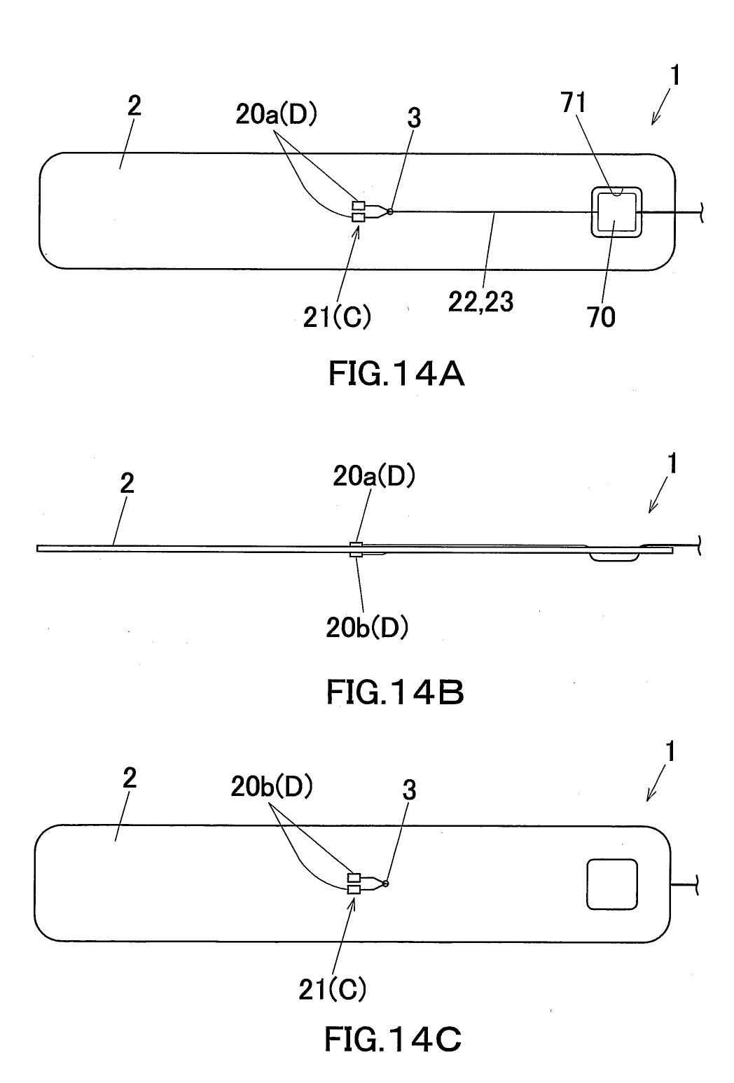

FIGS. 14A to 14C are explanatory views of a biological information measuring panel 1 according to the third fourth embodiment of this invention.

In the actual use, in the same manner as in the panel of the first embodiment, elastic layers are attached to the upper and lower surfaces of the laying plate portion 2 of this panel 1. However, in these figures, such elastic layers are not illustrated for an easy understanding of the structure of the elastic layer.

This panel 1 is provided with a controlling device 70 for controlling the output signal from the strain gauges 20a and 20b as a strain detecting sensor D. The controlling device 70 is attached on the substrate.

This controlling device 70 has an A/D converter for converting the output signal (e.g., output voltage) from the strain gauges 20a and 20b into a digital signal. This controlling device 70 is fixed to the laying plate portion 2 with adhesive agent or screws in a state in which the device 70 is disposed within a dented portion 71 formed at an end portion of the upper surface (or the lower surface) of the laying plate portion 2.

In this panel 1, since the controlling device 70 is attached to the laying plate portion 2, the output signal from the strain gauges 20a and 20b can be controlled by the controlling device 70.

Furthermore, since the controlling device 70 is disposed within the dented portion 71, damages due to the outside contacts of the controlling device 70 can be prevented. Therefore, the lifetime of the panel 1 can be extended.

Another constitutions of the panel of this fourth embodiment are the same as those of the aforementioned first embodiment.

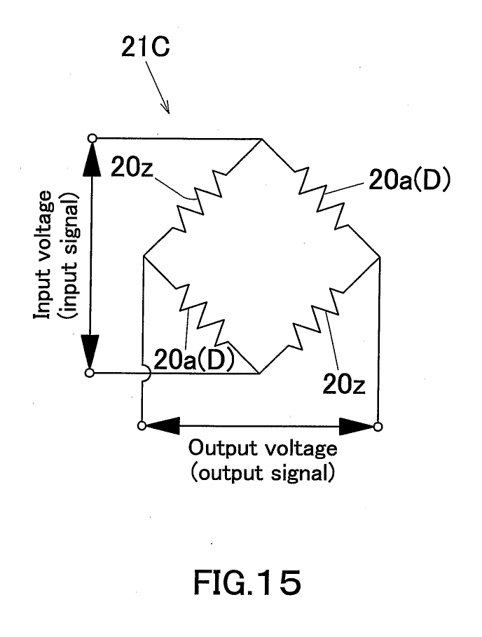

FIG. 15 shows a modified embodiment of the bridged circuit 21C used in this panel 1 of this fourth embodiment. In this bridged circuit 21C, a pair of strain gauges 20a and 20a are attached to the opposing portions of the upper and lower surfaces of the laying plate portion 2. On the other hand, the controlling portion 70 has fixed resistances 20Z and 20Z a pair of dummy resistance. This bridged circuit 21C is formed Z by electrically connecting the pair of strain gauges 20a and 20a and the pair of fixed resistances 20z and 20z.

With this bridged circuit 21C, the strain changes of the laying plate portion 2 can be detected assuredly.

In this invention, as a dummy resistance, chip resistances can be used in place of the fixed resistances 20z and 20z.

Although several embodiments of this invention have been explained, the present invention is not limited to the embodiments, and can be modified in various manner.

For example, in any of the aforementioned embodiments, strain gauges are attached to the upper and lower surfaces of the laying plate portion 2 of the panel 1. However, in this invention, the strain gauges can be attached only on the upper surface of the laying plate portion 2, or only on the lower surface of the laying plate portion 2.

The number of the strain gauges to be attached to the upper or lower surface of the laying plate portion 2 of the panel 1 can be one, two, three, four or more.

Furthermore, the strain gauges can be attached to a prescribed portion of the laying plate portion 2 by a mechanical securing means such as caulking, screwing, riveting, welding (e.g., spot welding) other than a means using adhesive agent.

The laying plate portion 2 of the panel 1 can be a member capable of receiving the entire subject S.

Furthermore, in this invention, the number of strain gauge group can be one, two, three, four or more.

Furthermore, in this embodiment, all of the laying plate portions 2 are disposed on the mat 17 of the bed 15, but the laying plate portion 2 can be dispose on a tatami or a floor.

Furthermore, in this invention, the biological information measuring panel, the mat or the biological information measuring device can be equipped with a safety device if necessary, or any safety processing can be made to these devices.

### INDUSTRIAL APPLICABILITY

This invention can be applied a biological information measuring panel used in measuring biological information of a subject such as a healthy person, a sick person, an infant or an aged person in a medical care facility, nursing care facility or a general home, a biological information measuring mat with the panel, a biological information measuring device with the panel or the mat, and a biological information measuring method using the panel or the mat.

This invention can also be applied to a system for monitoring biological activities of a subject.

While the present invention may be embodied in many different forms, a number of illustrative embodiments are described herein with the understanding that the present disclosure is to be considered as providing examples of the principles of the invention and such examples are not intended to limit the invention to preferred embodiments described herein and/or illustrated herein.

While illustrative embodiments of the invention have been described herein, the present invention is not limited to the various preferred embodiments described herein, but includes any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to examples described in the present specification or during the prosecution of the application, which examples are to be construed as non-exclusive. For example, in the present disclosure, the term “preferably” is non-exclusive and means “preferably, but not limited to.” In this disclosure and during the prosecution of this application, means-plus-function or step-plus-function limitations will only be employed where for a specific claim limitation all of the following conditions are present in that limitation: a) “means for” or “step for” is expressly recited; b) a corresponding function is expressly recited; and c) structure, material or acts that support that structure are not recited. In this disclosure and during the prosecution of this application, the terminology “present invention” or “invention” may be used as a reference to one or more aspect within the present disclosure. The language present invention or invention should not be improperly interpreted as an identification of criticality, should not be improperly interpreted as applying across all aspects or embodiments (i.e., it should be understood that the present invention has a number of aspects and embodiments), and should not be improperly interpreted as limiting the scope of the application or claims. In this disclosure and during the prosecution of this application, the terminology “embodiment” can be used to describe any aspect, feature, process or step, any combination thereof, and/or any portion thereof, etc. In some examples, various embodiments may include overlapping features. In this disclosure and during the prosecution of this case, the following abbreviated terminology may be employed: “e.g.” which means “for example;” and “NB” which means “note well.”

1. A biological information measuring panel, comprising:
an elastically flexible laying plate portion to be disposed below a subject; and
a strain detecting sensor attached to the laying plate portion,
wherein strain changes of the laying plate portion generated in accordance with biological activities of the subject are detected by the strain detecting sensor in a state in which the laying plate portion is disposed below the subject, and
wherein an output signal from the strain detecting sensor is used to measure the biological information of the subject.

2. The biological information measuring panel as recited in claim 1, wherein the laying plate portion is formed into an approximately rectangular belt shape and is to be disposed below the subject so as to extend in a widthwise direction of the subject.

3. The biological information measuring panel as recited in claim 1, wherein bending strength of the laying plate portion is set so as to fall within the range of from 7.5×10² to 1.5×10¹² Nmm².

4. The biological information measuring panel as recited in claim 1, wherein the laying plate portion is made of a material whose Young's modulus falls within the range of from 3×10⁴ to 30×10⁴ MPa.

5. The biological information measuring panel as recited in claim 1, wherein the laying plate portion is set to be 10 to 1,000 mm in length, 300 to 2,000 mm in width, and 0.1 to 30 mm in thickness.

6. The biological information measuring panel as recited in claim 1, wherein the laying plate portion is made of aluminum or its alloy.

7. The biological information measuring panel as recited in claim 1, further comprising an elastic layer attached to at least one of upper and lower surfaces of the laying plate portion.

8. The biological information measuring panel as recited in claim 7, wherein the elastic layer is an elastic sheet member, and wherein the elastic sheet member is attached to at least one of upper and lower surfaces of the laying plate portion with a peripheral end portion of the elastic sheet member protruded from a periphery of the laying plate portion.

9. The biological information measuring panel as recited in claim 1, wherein the strain detecting sensor is a strain gauge.

10. The biological information measuring panel as recited in claim 9, wherein at least one strain gauge is attached to at least one of the upper and lower surfaces of the laying plate portion.

11. The biological information measuring panel as recited in claim 9,
wherein a pair of strain gauges are attached to facing positions of the upper and lower surfaces of the laying plate portion,
wherein the pair of strain gauges and a pair of dummy resistances are electrically connected each other to form a bridged circuit, and
wherein an output signal from the bridged circuit is used to measure the biological information of the subject.

12. The biological information measuring panel as recited in claim 9,
wherein two pairs of strain gauges are attached to facing positions of the upper and lower surfaces of the laying plate portion,
wherein the two pairs of strain gauges are electrically connected each other to form a bridged circuit, and
wherein an output signal from the bridged circuit is used to measure the biological information of the subject.

13. The biological information measuring panel as recited in claim 9,
wherein the strain gauges are attached to right and left side portions and a central portion of at least one of the upper and lower surfaces of the laying plate portion.

14. The biological information measuring panel as recited in claim 9,
wherein the laying plate portion is provided with at least one thin thickness portion thinner than the other portion of the laying plate portion,
wherein at least one strain gauge is attached to at least one surface of the upper and lower surfaces of the thin thickness portion of the laying plate portion.

15. The biological information measuring panel as recited in claim 14,
wherein the thin thickness portion is formed in the laying plate portion by partially forming a dented portion on at least one of the upper and lower surfaces of the thin thickness portion of the laying plate portion.

16. The biological information measuring panel as recited in claim 14,
wherein the thin thickness portion is formed in the laying plate portion by forming the laying plate portion so that a thickness of the laying plate portion continuously changes in a widthwise direction of the laying plate portion.

17. The biological information measuring panel as recited in claim 9,
wherein a signal line passing opening for passing input signal lines to the strain gauges and/or output signal lines from the strain gauges is formed near a strain gauge attaching position.

18. The biological information measuring panel as recited in claim 1,
wherein the strain detecting sensor is a conductive elastomer sensor.

19. The biological information measuring panel as recited in claim 18,
wherein the elastomer sensor is disposed inside the laying plate portion.

20. The biological information measuring panel as recited in claim 18,
wherein the laying plate portion is divided into an upper plate portion and a lower plate portion, and wherein the upper plate portion and the lower plate portion are secured in a superimposed manner in a state in which the conductive elastomer sensor is sandwiched between the upper plate portion and the lower plate portion.

21. The biological information measuring panel as recited in claim 1,
further comprising a controlling device for controlling an output signal from the strain detecting sensor is attached to the laying plate portion.

22. The biological information measuring panel as recited in claim 21,
wherein the controlling device is disposed in a dented portion formed in the upper surface or the lower surface of the laying plate portion.

23. A biological information measuring mat, comprising:
the biological information measuring panel as recited in claim 1,
wherein the panel is disposed in the mat.

24. A biological information measuring device, comprising:
the biological information measuring panel as recited in claim 1 or the biological information measuring mat as recited in claim 23,
wherein biological information of the subject is measured based on an output signal from the strain detecting sensor of the panel or the mat.

25. The biological information measuring device as recited in claim 24, further comprising:
computing means for computing the biological information of the subject based on the output signal from the strain detecting sensor; and
displaying means for displaying the biological information computed by the computing means.

26. The biological information measuring device as recited in claim 25, further comprising communicating means configured to transmit the biological information computed by the computing means.

27. The biological information measuring device as recited in claim 25, further comprising warning means configured to give a warning based on the biological information computed by the computing means.

28. A biological information measuring method, comprising the steps of:
preparing the biological information measuring panel as recited in claim 1 or the biological information measuring mat as recited in claim 23,
detecting strain changes of the laying plate portion of the panel or the mat generated in accordance with biological activities of the subject in a state in which the panel or the mat is disposed below the subject, and
measuring the biological information of the subject based on an output signal from the strain detecting sensor.

29. The biological information measuring method as recited in claim 28,
wherein the biological information of the subject is computed by computing means based on the output signal from the strain detecting signal, and
wherein the biological information computed by the computing means is displayed by displaying means.

30. The biological information measuring method as recited in claim 29, wherein the biological information computed by the computing means is transmitted by communicating means.

31. The biological information measuring method as recited in claim 29, wherein a warning is given by warning means based on the biological information computed by the computing means.

32. A biological activities monitoring system, comprising:
the biological information measuring panel as recited in claim 1 or the biological information measuring mat as recited in claim 23;
computing means configured to compute the biological information of the subject based on an output signal from the strain detecting sensor of the panel or the mat;
displaying means configured to display the biological information computed by the computing means; and
communicating means configured to transmit the biological information computed by the computing means.
